(19) United States
(12) Patent Application Publication
(10) Pub. No.: US 2009/0220589 A1
(43) Pub. Date: Sep. 3, 2009

(54) TREATMENT, DIAGNOSTIC, AND METHOD FOR DISCOVERING ANTAGONIST USING SPARC SPECIFIC MIRNAS

(75) Inventors: Vuong Trieu, Calabasas, CA (US); Larn Hwang, Arcadia, CA (US); Neil Desai, Los Angeles, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Abraxis BioScience, LLC, Los Angeles, CA (US)

(21) Appl. No.: 12/395,872

(22) Filed: Mar. 2, 2009

Related U.S. Application Data
(60) Provisional application No. 61/032,961, filed on Mar. 1, 2008.

(51) Int. Cl. A61K 9/127; A61K 31/7105; C07H 21/02; C12N 15/63; A61P 35/00; C12Q 1/68; C07K 2/00

(52) U.S. Cl. 424/450; 536/24.5; 435/320.1; 536/24.1; 435/6; 530/322; 530/300; 514/44 A

(57) ABSTRACT

miRNAs that regulate human SPARC and methods of use thereof are described. Suitable nucleic acids for use in the methods and compositions described herein include, but are not limited to, pri-miRNA, pre-miRNA, ds miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA.

### CROSS-REFERENCE TO RELATED APPLICATIONS

This patent application claims the benefit of U.S. Provisional Patent Application No. 61/032,961, filed on Mar. 1, 2008, which is incorporated by reference in its entirety.

### BACKGROUND OF THE INVENTION

Secreted protein acidic and rich in cysteine (also known as osteonectin, BM40, or SPARC) (hereainfter “SPARC”), is a matrix-associated protein that elicits changes in cell shape, inhibits cell-cycle progression, and influences the synthesis of extracellular matrix (Bradshaw et al., Proc. Nat. Acad. Sci. USA 100: 6045-6050 (2003)). The murine SPARC gene was cloned in 1986 (Mason et al., EMBO J. 5: 1465-1472 (1986)) and a full-length human SPARC cDNA (SEQ ID NO: 1) was cloned and sequenced in 1987 (Swaroop et al., Genomics 2: 37-47 (1988)). SPARC expression is developmentally regulated, and is predominantly expressed in tissues undergoing remodeling during normal development or in response to injury. For example, high levels of SPARC protein are expressed in developing bones and teeth (see, e.g., Lane et al., FASEB J., 8, 163 173 (1994); Yan & Sage, J. Histochem. Cytochem. 47:1495-1505 (1999)).

SPARC is upregulated in several aggressive cancers, but is absent in the corresponding normal tissues (e.g., bladder, liver, ovary, kidney, gut, and breast) (Porter et al., J. Histochem. Cytochem., 43, 791 (1995)). In bladder cancer, for example, SPARC expression has been associated with advanced carcinoma. Invasive bladder tumors of stage T2 or greater have been shown to express higher levels of SPARC relative to bladder tumors of stage T1 (or less superficial tumors), and poorer prognosis (see, e.g., Yamanaka et al., J. Urology, 166, 2495 2499 (2001)). In meningiomas, SPARC expression has been associated only with invasive tumors (see, e.g., Rempel et al., Clincal Cancer Res., 5, 237 241 (1999)). SPARC expression also has been detected in 74.5% of in situ invasive breast carcinoma lesions (see, e.g., Bellahcene, et al., Am. J. Pathol., 146, 95 100 (1995)), and 54.2% of infiltrating ductal carcinoma of the breast (see, e.g., Kim et al., J. Korean Med. Sci., 13, 652 657 (1998)). SPARC expression also has been associated with frequent microcalcification in breast cancer (see, e.g., Bellahcene et al., supra), suggesting that SPARC expression may be responsible for the affinity of breast metastases for the bone.

Surprisingly, SPARC has also been reported to be markedly down-regulated in ovarian carcinomas relative to the normal surface epithelium and has been suggested to act as a tumor suppressor in ovarian cancer (Brown, T. J. et al., Gynecol Oncol. (1999) 75(1):25-33). Accordingly, SPARC has been shown to have anti-tumor activity in some model systems. SPARC is a potent cell cycle inhibitor that arrests cells in mid-G1 (Yan & Sage, J. Histochem. Cytochem. 47:1495-1505 (1999)) and the inducible expression of SPARC has been shown to inhibit breast cancer cell proliferation in an in vitro model system (Dhanesuan et al., Breast Cancer Res. Treat. 75:73-85 (2002)). Similarly, exogenous SPARC can reduce the proliferation of both HOSE (human ovarian surface epithelial) and ovarian cancer cells in a concentration-dependent manner. In addition, SPARC induces apoptosis in ovarian cancer cells. Further evidence for SPARC receptors present on cells such as ovarian epithelial cells has been report. It has been proposed that the binding of SPARC to its receptor is likely to trigger tissue-specific signaling pathways that mediate its tumor suppressing functions (Yiu et al., Am. J. Pathol. 159:609-622 (2001)). Purified SPARC has also been reported to potently inhibit angiogenesis and significantly impair neuroblastoma tumor growth in an in vivo xenograft model system (Chlenski et al., Cancer Res. 62:7357-7363 (2002)).

SPARC also plays a role in non-neoplastic proliferative diseases. Mesangial cell proliferation is a characteristic feature of many glomerular diseases and often precedes extracellular matrix expansion and glomerulosclerosis. In a model of experimental mesangioproliferative glomerulonephritis, SPARC mRNA was increased 5-fold by day 7 and was identified in the mesangium by in situ hybridization. However, recombinant SPARC or a synthetic SPARC peptide inhibited platelet-derived-growth-factor-induced mesangial cell DNA synthesis in vitro (Pichler et al., Am. J. Pathol. 148(4):1153-67 (1996)). Similarly, while renal enlargement, due to hyperplasia, hypertrophy, and increase inter-cellular matrix, is a characteristic feature of diabetes in humans, kidney SPARC mRNA levels fell in diabetic animals. In addition, the onset of diabetes-related kidney growth is associated with a reduction in SPARC mRNA and protein (Gilbert et al., Kidney Int. 48(4):1216-25 (1995)).

SPARC has been implicated in the pathogenesis of atherosclerotic lesions. Plasma SPARC levels are elevated in patients with coronary artery disease (Masahiko et al., Obesity Res. 9:388-393 (2001)). The proliferation of vascular smooth muscle cells in the arterial intima plays a central role in the pathogenesis of atherosclerosis. SPARC is expressed in vascular smooth muscle cells and macrophages associated with atherosclerotic lesions. In addition, SPARC has been hypothesized to regulate the action of platelet-derived growth factor during vascular injury (Masahiko et al., Obesity Res. 9:388-393 (2001); Raines et al., Proc. Natl. Acad. Sci. USA 89:1281-1285 (1992)). A stimulatory effect of SPARC on endothelial PAI-1 production has been reported at the site of vascular injury (Hasselaar et al., J. Biol. Chem. 266:13178-13184 (1991)) and has been postulated to accelerate atherosclerosis (Masahiko et al., Obesity Res. 9:388-393 (2001))

SPARC has affinity for a wide variety of ligands including cations (e.g., Ca 2+, Cu 2+, Fe 2+), growth factors (e.g., platelet derived growth factor (PDGF), and vascular endothelial growth factor (VEGF)), extracellular matrix (ECM) proteins (e.g., collagen I V and collagen IX, vitronectin, and thrombospondin 1), endothelial cells, platelets, albumin, and hydroxyapaptite (see, e.g., Lane et al., FASEB J., 8, 163 173 (1994); Yan & Sage, J. Histochem. Cytochem. 47:1495-1505 (1999)). SPARC is also known to bind albumin (see, e.g., Schnitzer, J. Biol. Chem., 269, 6072 (1994)).

It is therefore an object of the present invention to provide naturally occurring miRNAs for inhibition of expression of SPARC where SPARC overexpression has been shown to be associated with poor prognosis and/or causal of the disease. The invention is based on the demonstration of 3′UTR inhibition of SPARC mRNA translation indicative of miRNA translational inhibition.

It is further an object of the present invention to provide naturally occurring nucleic acids for treatment or prophylaxis of one or more symptoms of cancer or proliferative diseases which are dependent on or caused by SPARC under-expression.

### BRIEF SUMMARY OF THE INVENTION

The invention provides methods for inhibiting the expression of SPARC protein comprising administering to an organism, such as a human patient afflicted with, e.g. cancer, restenosis or other cellular proliferative disease or condition, an inhibitorily effective amount of one or more miRNAs that bind to endogenous SPARC RNA and inhibit SPARC protein expression in the cells of an organism. Further, miRNAs in accordance with the invention include synthetic RNAs and miRNAs encoded and expressed from isolated nucleic acids genetically engineered for the expression of the miRNA in the cells of an organism.

Accordingly, the invention also provides therapeutic compositions for administration to a patient in need of the treatment or prevention of cancer, restenosis or other proliferative disease comprising a synthetic RNA or an isolated nucleic acid for the expression in the cells of the patient of an effective amount of miRNA to bind to SPARC mRNA and inhibit expression of SPARC protein.

In addition, the invention further provides methods for increasing the expression SPARC protein in the cells of an organism comprising administering to the organism an effective amount of one or more antagonists that bind to one or more endogenous miRNAs and reverse the inhibition of SPARC protein expression by the endogenous miRNA .

The invention further provides therapeutic compositions for administration to a patient in need of the treatment or prevention of cancer, restenosis or other proliferative disease comprising synthetic RNAs and isolated nucleic acids for the expression in the cells of the patient an effective amount of one or more antagonistix miRNAs that bind to one or more endogenous miRNAs so as to reverse the inhibition of SPARC protein expression by the endogenous miRNA. The antagonist can be any suitable synthetic nucleic acid, including, e.g., an RNA, DNA, PNA (peptide nucleic acid), LNA (locked nucleic acid) or derivatives thereof. Alternatively, the antagonist can be encoded by and expressed from an isolated nucleic acid.

In particularly preferred embodiments of the invention the miRNA target sequence is SPARC (SEQ ID NO: 1) and miRNA sequences or their complements are selected from the group consisting: hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-let-7d, hsa-miR-29a, hsa-miR-29b, hsa-mir-29c, hsa-let-7g, hsa-miR-146b-5p, hsa-miR-154, hsa-miR-674, hsa-let-7f, hsa-miR-21, hsa-miR-22, hsa-miR-23a, hsa-miR-98, hsa-let-7a, hsa-miR-199a-3p, hsa-miR-214, hsa-miR-130a, hsa-miR-211, hsa-miR-515-5p, hsa-miR-517a, hsa-miR-517b, hsa-mir-203 ( in their stem-loop form SEQ ID NOS: 2-35, or the mature sequences SEQ ID NOS: 44-83, respectively), as well as SEQ ID NOS: 36-41 and 84-89 and combinations thereof. In their mature form these miRNAs have the following sequences:

| | | | |
|---|---|---|---|
| miRNA | SEQ ID NO. | Mature Accession | Mature sequence |

| | | | |
|---|---|---|---|
| hsa-miR-885-5p | 44 | MIMAT0004947 | UCCAUUACACUACCCUGCCUCU |
| hsa-let-7b | 45 | MIMAT0000063 | UGAGGUAGUAGGUUGUGUGGUU |
| hsa-let-7i | 46 | MIMAT0000415 | UGAGGUAGUAGUUUGUGCUGUU |
| hsa-miR-186 | 47 | MIMAT0000456 | CAAAGAAUUCUCCUUUUGGGCU |
| hsa-miR-125b | 48 | MIMAT0000423 | UCCCUGAGACCCUAACUUGUGA |
| hsa-let-7d | 49 | MIMAT0000065 | AGAGGUAGUAGGUUGCAUAGUU |
| hsa-miR-34c-5p | 50 | MIMAT0000686 | AGGCAGUGUAGUUAGCUGAUUGC |
| hsa-miR-139-5p | 51 | MIMAT0000250 | UCUACAGUGCACGUGUCUCCAG |
| hsa-miR-100 | 52 | MIMAT0000098 | AACCCGUAGAUCCGAACUUGUG |
| hsa-miR-34b | 53 | M1MAT0004676 | CAAUCACUAACUCCACUGCCAU |
| hsa-let-7c | 54 | MIMAT0000064 | UGAGGUAGUAGGUUGUAUGGUU |
| hsa-miR-29a | 55 | MIMAT0000086 | UAGCACCAUCUGAAAUCGGUUA |
| hsa-miR-29a* | 56 | MIMAT0004503 | ACUGAUUUCUUUUGGUGUUCAG |
| hsa-mir-29b-1 | 57 | MIMAT0000100 | UAGCACCAUUUGAAAUCAGUGUU |
| hsa-mir-29b-2 | 58 | MIMAT0000100 | UAGCACCAUUUGAAAUCAGUGUU |
| hsa-mir-29c | 59 | MIMAT0000681 | UAGCACCAUUUGAAAUCGGUUA |
| hsa-let-7g | 60 | MIMAT0000414 | UGAGGUAGUAGUUUGUACAGUU |
| hsa-miR-146b-5p | 61 | MIMAT0002809 | UGAGAACUGAAUUCCAUAGGCU |
| hsa-miR-154 | 62 | MIMAT0000452 | UAGGUUAUCCGUGUUGCCUUCG |
| hsa-miR-674 | 63 | MIMAT0003740 | GCACUGAGAUGGGAGUGGUGUA |
| (mmu-miR-674) | | | |
| hsa-let-7f | 64 | MIMAT0000067 | UGAGGUAGUAGAUUGUAUAGUU |
| hsa-miR-21 | 65 | MIMAT0000076 | UAGCUUAUCAGACUGAUGUUGA |
| hsa-miR-22 | 66 | MIMAT0000077 | AAGCUGCCAGUUGAAGAACUGU |
| hsa-miR-23a | 67 | MIMAT0000078 | AUCACAUUGCCAGGGAUUUCC |
| hsa-miR-98 | 68 | MIMAT0000096 | UGAGGUAGUAAGUUGUAUUGUU |
| hsa-let-7a | 69 | MIMAT0000062 | UGAGGUAGUAGGUUGUAUAGUU |
| hsa-miR-199a-3p | 70 | MIMAT0000232 | ACAGUAGUCUGCACAUUGGUUA |
| hsa-miR-214 | 71 | MIMAT0000271 | ACAGCAGGCACAGACAGGCAGU |
| hsa-miR-130a | 72 | MIMAT0000425 | CAGUGCAAUGUUAAAAGGGCAU |
| hsa-miR-211 | 73 | MIMAT0000268 | UUCCCUUUGUCAUCCUUCGCCU |
| hsa-miR-515-5p | 74 | MIMAT0002826 | UUCUCCAAAAGAAAGCACUUUCUG |
| hsa-miR-517a | 75 | MIMAT0002852 | AUCGUGCAUCCCUUUAGAGUGU |
| hsa-miR-517b | 76 | MIMAT0002857 | UCGUGCAUCCCUUUAGAGUGUU |
| hsa-mir-203 | 77 | MIMAT0000264 | GUGAAAUGUUUAGGACCACUAG |
| hsa-miR-297 | 78 | MIMAT0004450 | AUGUAUGUGUGCAUGUGCAUG |
| hsa-mir-573 | 79 | MIMAT0003238 | CUGAAGUGAUGUGUAACUGAUCAG |
| hsa-mir-758 | 80 | MIMAT0003879 | UUUGUGACCUGGUCCACUAACC |
| hsa-mir-583 | 81 | MIMAT0003248 | CAAAGAGGAAGGUCCCAUUAC |
| hsa-mir-7 | 82 | MIMAT0000252 | UGGAAGACUAGUGAUUUUGUUGU |
| hsa-mir-1 | 83 | MIMAT0000416 | UGGAAUGUAAAGAAGUAUGUAU |

In other embodiments, the invention provides an isolated nucleic acid comprising one or more in vivo expression control elements operatively linked to a reporter gene, wherein said reporter gene is upstream of all or a portion of a SPARC RNA's 3′ untranslated region, wherein upon transfection of the isolated nucleic acid into eukaryotic cells, the in vivo expression control elements result the production of an mRNA encoding the reporter upstream of the SPARC 3′ untranslated region.

Accordingly, the invention further provides a kit for the identification of SPARC expression modulators comprising:

(a) first isolated nucleic acid with a first set of one or more in vivo expression control elements operatively linked to a first reporter gene which is cloned upstream of all or a portion of a SPARC 3′ untranslated region, wherein upon transfection of said first isolated nucleic acid into eukaryotic cells, the first set of in vivo expression control elements result the production of an mRNA encoding the first reporter upstream of the SPARC 3′ untranslated region; (b) a second isolated nucleic acid comprising said the set of in vivo expression control elements from (a) operatively linked to said first reporter gene, wherein upon transfection of said second isolated nucleic acid into eukaryotic cells, the in vivo expression control elements result in the transcription of an mRNA encoding said first reporter molecule; and (c) a third isolated nucleic acid comprising a second set of one or more in vivo expression control elements operatively linked to a second reporter gene, wherein upon transfection of the isolated nucleic acid into eukaryotic cells, said second set of in vivo expression control elements result in the expression of said second reporter.

Thus, the invention provides methods of identifying SPARC expression modulators comprising: (a) transfecting eukaryotic cells with an isolated nucleic acid comprising one or more in vivo expression control elements operatively linked to a reporter gene which is cloned upstream of all or a portion of a SPARC 3′ untranslated region, wherein the in vivo expression control elements result the production of an mRNA encoding the reporter upstream of the SPARC 3′ untranslated region, and (b) transfecting other eukaryotic cells with isolated nucleic acid comprising said one or more in vivo expression control elements operatively linked to said reporter gene, wherein the expression control elements result in the transcription of an mRNA encoding the reporter molecule, (c) contacting and mock-contacting the transfected cells from (a) and (b) with a candidate expression modulator, and (d) comparing the reporter gene activity in the transfected cells from (a) and (b) with and without contacting the transfected cells with candidate expression modulator.

The methods of identifying SPARC expression modulators provided by the invention can additionally comprise the co-transfection of the cells in (a) and (b), with a second report construct expressing a second reporter for the normalization the data compared in (d). This method identifying SPARC expression modulators can further mutating the SPARC 3′ untranslated region in the reporter expression construct, transfecting said mutated reporter expression construct into eukaryotic cells, and comparing the reporter gene activity resulting from expression of the mutated and unmutated reporter expression constructs with and without contacting the transfected cells with candidate expression modulator. The SPARC expression modulator so identified can be, e.g., a small molecule, nucleic acid, peptide-nucleic acid, miRNA or a polypeptide.

The invention also provides methods of inhibiting the expression of one or more proteins in the cells of an organism, wherein said proteins are selected from the group consisting of clusterin, β chain; clusterin, α chain; N-cadherin; secemin 1; collagen, type v, α-chain; renin, βchain; renin; and cytokeratin I, type II, and of increasing the expression of one or more proteins in the cells of an organism, wherein said proteins are selected from the group consisting of α-actin; hsp27; collagen, type I, α-2 chain; peroxiredoxin 3; β-5 tubulin; p32, chain said method comprising comprising administering the organism an inhibitorily effective amount of one or more miRNAs that bind to and inhibit SPARC expression in the cells of the organism.

Further, the invention provides methods of modulating the expression of one or more proteins in the cells of an organism, wherein said proteins are encoded by nucleic acid sequences selected from the group consisting of the following Genebank accession numbers: Human mRNAs: NM_{—}016619, NM_{—}016323, NM_{—}012294, NM_{—}006393, NM_{—}005609, NM_{—}002462, NM_{—}002346, NM_{—}001955, NM_{—}001548, NM_{—}000909, BM930167, BM874773, B1560717, AW511255, AK098543, A1860360, A1760944; human counterpart of the following mouse mRNAs: NM_{—}133664, NM_{—}011641, NM_{—}010226, NM_{—}008380, BB480262, AW909062; human miRNAs: hsa-miR-542-5p, hsa-miR-186; human counterparts of the following mouse miRNAs: rno-miR-377, mmu-mir-377 comprising administering to the organism an effective amount of one or more miRNAs that bind to and inhibit SPARC expression in the cells of the organism.

The invention finally provides for the use of these miRNA as biomarker for proliferative disease progression, response to a treatment of proliferative disease or combinations thereof.

### BRIEF DESCRIPTION OF THE SEVERAL VIEWS OF THE DRAWINGS

FIG. 1 shows a Western blot detecting the expression of exogenous SPARC in 293 and CHO at 48 hours posttransfection.

FIG. 2 shows a Western blot detecting the expression of exogenous SPARC in stably transfected 293 and CHO cells at 3 weeks posttransfection.

FIG. 3 depicts the location of primers used to detect SPARC mRNAs.

FIG. 4 shows gel electrophoresis of RT-PCT products from exogenous and endogenous mRNAs encoding SPARC in CHO and 293 cells at 48 hours post-transfection.

FIG. 5 shows a Western blot detecting the expression of exogenous SPARC in CHO and 293 cells transfected with the LH-3-6×His or LH-3-6×His-3′UTR constructs.

FIG. 6 depicts a restriction map of pXL-miRNA1.1 with SPARC 3′UTR cloned immediately downstream of the coding region for luciferase.

FIG. 7 shows normalized luciferase expression from CHO and 293 cells transfected a construct with SPARC 3′UTR cloned immediately downstream of the coding region for luciferase.

FIG. 8 depicts luciferase expression from constructs without (pMIR-reporter) or with the different versions of the SPARC's 3′UTR (pXL-mRNA1.1; pXL-mRNA1.2) in SK-OV-3, Caov-3, ES-2, PA-1, and OVCAR-3 cells.

FIG. 9 shows the result of screening a pre-miRNA library with the Luciferase-SPARC 3′UTR reporter system.

FIG. 10 shows microarray results comparing CHO and 293 miRNAs.

### DETAILED DESCRIPTION OF THE INVENTION

I. Definitions

As used herein the term “SPARC protein” refers to a polypeptide of with an identical sequence to either the unprocessed (SEQ ID NO: 42) or mature SPARC polypeptide (SEQ ID NO: 43) or a natural spice variant generated from SEQ ID NO: 41 or a polypeptide of substantially the identical sequence to either SEQ ID NO: 42 or 43 and which substantially retains the function of the mature SPARC polypeptide. By “a substantially the identical sequence” it is meant that the sequence is at least 80% identical, preferably at least 85% identical, more preferably at least 90% identical, even more preferably at least 95% identical, and most preferably at least 99% identical to either SEQ ID NOS: 42 or 43. By “substantially retains the function of the mature SPARC” it is meant that the polypeptide has one or more of the biological/biochemical activities of SPARC known to those of ordinary skill, particularly activities that effect (maintain, support, induce, cause, diminish, prevent or inhibit) a disease state, including, e.g., influencing angiogenesis, cell shape, cell motility, cell adhesion, apoptosis, cellular proliferation or the composition of the extracellular matrix. An example of a polypeptide of substantially the identical sequence to either SEQ ID NO: 42 or 43 and which substantially retains the function of the mature SPARC polypeptide is the SPARC Q3 mutant disclosed in U.S. Pat. No. 7,332,568. Said polypeptides encompassed by the term “SPARC protein” also include polypeptides which have about 50 amino acids, preferably about 40 amino acids, more preferably about 30 amino acids, even more preferably about 20 amino acids, and most preferably about 10 amino acids added to the amino and/or carboxyl termini of a sequence that is identical to or substantially identical to SEQ ID NOS: 42 or 43.

As used herein the term “endogenous SPARC RNA” refers to an RNA molecule comprised of the coding sequence of a SPARC protein.

As used herein the term “inhibitorily effective” refers to a result which substantially decreases the level or expression of, including for example, an about 20% reduction, preferrably an about 25% reduction, more preferrably an about 33% reduction, even more preferrably an about 50% reduction, even more preferrably an about 67% reduction, even more preferrably an about 80% reduction, even more preferrably an about 90% reduction, even more preferrably an about 95% reduction, even more preferrably an about 99% reduction, even more preferrably an about 50 fold reduction, even more preferrably an about 100 fold reduction, even more preferrably an about 1,000 fold reduction, even more preferrably an about 10,000 fold reduction, and most preferable complete silencing.

Similarly, an “effective amount” is an amount that would produce changes of the same magnitude as that of an “inhibitorily effective amount,” but in any desired direction of up or down regulation.

As used herein the term “reducing reporter activity” refers to a result which substantially decreases the level of expression or activity, including for example, an about 20% reduction, preferrably an about 25% reduction, more preferrably an about 33% reduction, even more preferrably an about 50% reduction, even more preferrably an about 67% reduction, even more preferrably an about 80% reduction, even more preferrably an about 90% reduction, even more preferrably an about 95% reduction, even more preferrably an about 99% reduction, even more preferrably an about 50 fold reduction, even more preferrably an about 100 fold reduction, even more preferrably an about 1,000 fold reduction, even more preferrably an about 10,000 fold reduction, and most preferable complete silencing.

As used herein the term “nucleic acid” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars. In addition, as used herein, the term “nucleic acid” includes peptide nucleic acids.

As used herein, the term “microRNA” refers to any type of interfering RNA, including but not limited to, endogenous microRNA and artificial microRNA. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA.

“MicroRNA flanking sequence” as used herein refers to nucleotide sequences including microRNA processing elements. MicroRNA processing elements are the minimal nucleic acid sequences which contribute to the production of mature microRNA from precursor microRNA. Precursor miRNA termed pri-miRNAs are processed in the nucleus into about 70 nucleotide pre-miRNAs, which fold into imperfect stem-loop structures. The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. Preferred structures have flanking sequences on both ends of the stem-loop structure. The flanking sequences may be directly adjacent to one or both ends of the stem-loop structure or may be connected to the stem-loop structure through a linker, additional nucleotides or other molecules.

As used herein a “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures and terms are well known in the art. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base-pairing may not include any mismatches.

II. miRNRAs

As used herein, the term miRNA includes, e.g., the following group of miRNAs: hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-let-7d, hsa-miR-29a, hsa-let-7g, hsa-miR-146b-5p, hsa-miR-154, hsa-miR-674, hsa-let-7f, hsa-miR-21, hsa-miR-22, hsa-miR-23a, hsa-miR-98, hsa-let-7a, hsa-miR-199a-3p, hsa-miR-214, hsa-miR-130a, hsa-miR-211, hsa-miR-515-5p, hsa-miR-517a, hsa-miR-517b, hsa-mir-29 b, has-mir-29c, hsa-miR-297, hsa-mir-573, hsa-mir-758, hsa-mir-583, hsa-mir-7, hsa-mir-1, and hsa-mir-203.1. In either their hairpin or mature forms.

The following table shows miRNAs with their database and SEQ ID NOS:

| | Hairpin-Sanger | SEQ ID | Mature Accession | SEQ ID |
|---|---|---|---|---|
| SiRNA Name | Accession No. | NO: | No. | NO: |
| hsa-miR-885-5p | MI0005560 | 2 | MIMAT0004947 | 44 |
| hsa-let-7b | MI0000063 | 3 | MIMAT0000063 | 45 |
| hsa-let-7i | MI0000434 | 4 | MIMAT0000415 | 46 |
| hsa-miR-186 | MI0000483 | 5 | MIMAT0000456 | 47 |
| hsa-miR-125b | MI0000470 | 6 | MIMAT0000423 | 48 |
| hsa-let-7d | MI0000065 | 7 | MIMAT0000065 | 49 |
| hsa-miR-34c-5p | MI0000743 | 8 | MIMAT0000686 | 50 |
| hsa-miR-139-5p | MI0000261 | 9 | MIMAT0000250 | 51 |
| hsa-miR-100 | MI0000102 | 10 | MIMAT0000098 | 52 |
| hsa-miR-34b | MI0000742 | 11 | MIMAT0004676 | 53 |
| hsa-let-7c | MI0000064 | 12 | MIMAT0000064 | 54 |
| hsa-miR-29a | MI0000087 | 13 | MIMAT0000086 | 55 |
| hsa-miR-29a* | MI0000087 | 14 | MIMAT0004503 | 56 |
| hsa-mir-29b-1 | MI0000105 | 15 | MIMAT0000100 | 57 |
| hsa-mir-29b-2 | MI0000107 | 16 | MIMAT0000100 | 58 |
| hsa-mir-29c | MI0000735 | 17 | MIMAT0000681 | 59 |
| hsa-let-7g | MI0000433 | 18 | MIMAT0000414 | 60 |
| hsa-miR-146b-5p | MI0003129 | 19 | MIMAT0002809 | 61 |
| hsa-miR-154 | MI0000480 | 20 | MIMAT0000452 | 62 |
| hsa-miR-674 | MI0004611 | 21 | MIMAT0003740 | 63 |
| (mmu-miR-674) | | | | |
| hsa-let-7f | MI0000067 | 22 | MIMAT0000067 | 64 |
| hsa-miR-21 | MI0000077 | 23 | MIMAT0000076 | 65 |
| hsa-miR-22 | MI0000078 | 24 | MIMAT0000077 | 66 |
| hsa-miR-23a | MI0000079 | 25 | MIMAT0000078 | 67 |
| hsa-miR-98 | MI0000100 | 26 | MIMAT0000096 | 68 |
| hsa-let-7a | MI0000060 | 27 | MIMAT0000062 | 69 |
| hsa-miR-199a-3p | MI0000281 | 28 | MIMAT0000232 | 70 |
| hsa-miR-214 | MI0000290 | 29 | MIMAT0000271 | 71 |
| hsa-miR-130a | MI0000448 | 30 | MIMAT0000425 | 72 |
| hsa-miR-211 | MI0000287 | 31 | MIMAT0000268 | 73 |
| hsa-miR-515-5p | MI0003147 | 32 | MIMAT0002826 | 74 |
| hsa-miR-517a | MI0003161 | 33 | MIMAT0002852 | 75 |
| hsa-miR-517b | MI0003165 | 34 | MIMAT0002857 | 76 |
| hsa-mir-203 | MI0000283 | 35 | MIMAT0000264 | 77 |
| hsa-miR-297 | MI0005775 | 36 | MIMAT0004450 | 78 |
| hsa-mir-573 | MI0003580 | 37 | MIMAT0003238 | 79 |
| hsa-mir-758 | MI0003757 | 38 | MIMAT0003879 | 80 |
| hsa-mir-583 | MI0003590 | 39 | MIMAT0003248 | 81 |
| hsa-mir-7 | MI0000263 | 40 | MIMAT0000252 | 82 |
| hsa-mir-1 | MI0000437 | 41 | MIMAT0000416 | 83 |

Suitable sequence variants of miRNA for use in accordance with the invention include: substitutional, insertional or deletional variants. Insertions include 5′ and/or 3′ terminal fusions as well as intrasequence insertions of single or multiple residues. Insertions can also be introduced within the mature sequence. These, however, ordinarily will be smaller insertions than those at the 5′ or 3′ terminus, on the order of 1 to 4 residues, preferably 2 residues, most preferably 1 residue.

Insertional sequence variants of miRNA are those in which one or more residues are introduced into a predetermined site in the target miRNA. Most commonly insertional variants are fusions of nucleic acids at the 5′ or 3′ terminus of the miRNA.

Deletion variants are characterized by the removal of one or more residues from the miRNA sequence. These variants ordinarily are prepared by site specific mutagenesis of nucleotides in the DNA encoding miRNA, thereby producing DNA encoding the variant, and thereafter expressing the DNA in recombinant cell culture. However, variant miRNA fragments may be conveniently prepared by in vitro synthesis. The variants typically exhibit the same qualitative biological activity as the naturally-occurring analogue, although variants also are selected in order to modify the characteristics of miRNA.

Substitutional variants are those in which at least one residue sequence has been removed and a different residue inserted in its place. While the site for introducing a sequence variation is predetermined, the mutation per se need not be predetermined. For example, in order to optimize the performance of a mutation at a given site, random mutagenesis may be conducted at the target region and the expressed miRNA variants screened for the optimal combination of desired activity. Techniques for making substitution mutations at predetermined sites in DNA having a known sequence are well known.

Nucleotide substitutions are typically of single residues; insertions usually will be on the order of about from 1 to 10 residues; and deletions will range about from 1 to 30 residues. Deletions or insertions preferably are made in adjacent pairs; i.e. a deletion of 2 residues or insertion of 2 residues. Substitutions, deletion, insertions or any combination thereof may be combined to arrive at a final construct. Changes may be made to increase the activity of the miRNA, to increase its biological stability or half-life, and the like. All such modifications to the nucleotide sequences encoding such miRNA are encompassed.

An isolated nucleic acid or DNA is understood to mean chemically synthesized DNA, cDNA or genomic DNA with or without the 3′ and/or 5′ flanking regions. DNA encoding miRNA can be obtained from other sources by a) obtaining a cDNA library from cells containing mRNA, b) conducting hybridization analysis with labeled DNA encoding miRNA or fragments thereof in order to detect clones in the cDNA library containing homologous sequences, and c) analyzing the clones by restriction enzyme analysis and nucleic acid sequencing to identify full-length clones.

As used herein nucleic acids and/or nucleic acid sequences are “homologous” when they are derived, naturally or artificially, from a common ancestral nucleic acid or nucleic acid sequence. Homology is generally inferred from sequence identity between two or more nucleic acids or proteins (or sequences thereof). As used herein two nucleic acids and/or nucleic acid sequences, including miRNAs, are “identical” if they have the same nucleotide at each corresponding position in the two sequences, wherein for the purposes of this analysis uracil and thymidine are treated equivalently. Two sequences have a percent identity based on the number of identical nucleotides they share when the sequences are aligned by a suitable algorithm such as b12seq (Tatiana A. Tatusova, Thomas L. Madden (1999), “Blast 2 sequences—a new tool for comparing protein and nucleotide sequences”, FEMS Microbiol Lett. 174:247-250) which is publicly available through the National Center for Biotechnology Information. Two sequences are “complementary” if they can base pair at all nucleotides. The percent complementarity is based on the percent of nucleotides in each strand that can base pair with the other sequence when the sequences are aligned for base pairing.

The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence similarity percentages (e.g., BLASTN using default parameters) are generally available. Software for performing BLAST analyses is publicly available through the National Center for Biotechnology Information.

Micro RNAs (referred to as “miRNAs”) are small non-coding RNAs, belonging to a class of regulatory molecules found in plants and animals that control gene expression by binding to complementary sites on target messenger RNA (mRNA) transcripts. miRNAs are generated from large RNA precursors (termed pri-miRNAs) that are processed in the nucleus into approximately 70 nucleotide pre-miRNAs, which fold into imperfect stem-loop structures (Lee, Y., et al., Nature (2003) 425(6956):415-9). The pre-miRNAs undergo an additional processing step within the cytoplasm where mature miRNAs of 18-25 nucleotides in length are excised from one side of the pre-miRNA hairpin by an RNase III enzyme, Dicer (Hutvagner, G., et al., Science (2001) 12:12 and Grishok, A., et al., Cell (2001) 106(1):23-34). MiRNAs have been shown to regulate gene expression in two ways. First, miRNAs that bind to protein-coding mRNA sequences that are exactly complementary to the miRNA induce the RNA-mediated interference (RNAi) pathway. Messenger RNA targets are cleaved by ribonucleases in the RISC complex. This mechanism of miRNA-mediated gene silencing has been observed frequently in plants (Hamilton, A. J. and D. C. Baulcombe, Science (1999) 286(5441):950-2 and Reinhart, B. J., et al., MicroRNAs in plants. Genes and Dev. (2002) 16:1616-1626), but an example is known from animals (Yekta, S., I. H. Shih, and D. P. Bartel, Science (2004) 304(5670):594-6).

In the second mechanism, miRNAs that bind to imperfect complementary sites on messenger RNA transcripts direct gene regulation at the posttranscriptional level but do not cleave their mRNA targets. MiRNAs identified in both plants and animals use this mechanism to exert translational control of their gene targets (Bartel, D. P., Cell (2004) 116(2):281-97).

Surprisingly, we have shown that the SPARC mRNA 3′UTR can be a target for inhibiting SPARC gene expression by inhibiting its translation in various tumor cells including six different ovarian tumor lines. miRNA profiling has demonstrated that there are a group of miRNA upregulated in CHO (Chinese Hamster Ovary cells) and down regulated in 293 cells. These include hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-let-7d, hsa-miR-29a, hsa-let-7g, hsa-miR- 146b-5p, hsa-miR- 154, hsa-miR-674, hsa-let-7f, hsa-miR-21, hsa-miR-22, hsa-miR-23a, hsa-miR-98, hsa-let-7a, hsa-miR-199a-3p, hsa-miR-214, and hsa-miR-130a which exhibited more than 10 fold differential expression in CHO versus 293 (human embryonic kidney cells). Scanning for putative miRNA interaction site defined the following as possible miRNAs involving in SPARC regulation: hsa-miR-211, hsa-miR-515 -5p, hsa-miR-517a, hsa-miR-517b, hsa-mir-29, and hsa-mir-203.1. Additionally, screening of a pre-miRNA library defines a series of miRNAs capable of inhibiting SPARC expression; these include has-mir29a, has-mir-29b, has-mir-29c, miR-297, miR-573, let-7g, let-7f, miR-98, miR-758, let-7i, miR-34b, miR-583, miR-7, and miR-1. Therefore, up-regulating these specific microRNAs or providing analogous pharmaceutical compounds exogenously, should be effective cancer therapies for tumors resulting from activation or over-expression of these oncogenes. MiRNAs nucleic acids including pri-miRNA, pre-miRNA, ds miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA, referred to jointly as “miRNAs” unless otherwise stated, are described. In one embodiment, the size range of the miRNA can be from 10 nucleotides to 170 nucleotides, although miRNAs of up to 2000 nucleotides can be utilized. In a preferred embodiment the size range of the miRNA is from about 70 to about 170 nucleotides in length. In another preferred embodiment, mature miRNAs of from about 10 to about 50, more preferably from about 21 to about 25 nucleotides in length can be used.

RNA-induced silencing complex, or RISC, is a multi-protein siRNA complex which cleaves dsRNA (e.g., incoming viral) and binds short antisense RNA strands which are then able to bind complementary strands. When it finds the complementary strand, it activates RNase activity and cleaves the RNA. This process is important both in gene regulation by miRNAs and in defense against viral infections, which often use double-stranded RNA as an infectious vector.

miRNAs are useful as diagnostics and as therapeutics. The compositions are administered to a patient in need of treatment or prophylaxis of at least one symptom or manifestation (since disease can occur/progress in the absence of symptoms) of cancer/proliferative diseases. In one embodiment, the compositions are administered in an effective amount to inhibit expression of SPARC. Effective, safe dosages can be experimentally determined in model organisms and in human trials by methods well known to one of ordinary skill in the art. The compositions can be administered alone or in combination with adjuvant cancer therapy such as surgery, chemotherapy, radiotherapy, thermotherapy, immunotherapy, hormone therapy and laser therapy, to provide a beneficial effect, e.g. reduce tumor size, reduce cell proliferation of the tumor, inhibit angiogenesis, inhibit metastasis, or otherwise improve at least one symptom or manifestation of the disease.

The miRNAs disclosed herein are also useful as diagnostics. Any suitable method of detection of these miRNAs can be used such as by RT-PCR, miRNA microarray, and other conventional nucleic acid detection systems, for example, a panel of miRNA sequences can be established which can be predictive of, e.g., the response to chemotherapy, including Abraxane, or monitor the progression of the disease.

In some tumor types, SPARC underexpression instead of overexpression is associated with poor response. These patients include ovarian cancer and pancreatic cancer patients. Therefore, for these patient, there is a need for induction of SPARC. Also, overexpression of SPARC would be associated with improved tumor accumulation of nab-bound drugs as well as apoptotic death of cancer cells.

SPARC has been shown to be translationally regulated by miRNA in its 3′UTR. Therefore, up-regulating these specific microRNAs or providing analogous pharmaceutical compounds exogenously, should be effective for inhibition of SPARC expression and treatment of cancer or proliferative diseases associated with increased SPARC expression.

In preferred embodiments, the miRNA formulations are administered to individuals with a cancer that overexpressed SPARC.

Naturally occurring microRNAs that regulate human oncogenes, pri-miRNA, pre-miRNA, ds miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA, have been identified. The size of the miRNA is typically from about 11 nucleotides to about 170 nucleotides, although nucleotides of up to about 2000 nucleotides can be utilized. In a preferred embodiment the size range of the pre-miRNA is from about 70 to about 170 nucleotides in length and the mature miRNA is from about 10 to about 50, more preferably from about 21 to about 25 nucleotides in length.

The miRNA is selected from the group of miRNA shown to upregulated in ovarian lines exhibiting poor SPARC expression and down regulated in non-ovarian line shown to have strong SPARC expression. These include, e.g., the hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-let-7d, hsa-miR-29a, hsa-let-7g, hsa-miR-146b-5p, hsa-miR-154, hsa-miR-674, hsa-let-7f, hsa-miR-21, hsa-miR-22, hsa-miR-23a, hsa-miR-98, hsa-let-7a, hsa-miR-199a-3p, hsa-miR-214, hsa-miR-130a, hsa-miR-21 1, hsa-miR-515-5p, hsa-miR-517a, hsa-miR-517b, hsa-mir-29, and hsa-mir-203.1. miRNAs identified by computational analysis: hsa-miR-211, hsa-miR-515-5p, hsa-miR-517a, hsa-miR-517b, hsa-mir-29, and hsa-mir-203.1. miRNAs identified by screening against a pre-miRNA library: has-mir29a, has-mir-29b, has-mir-29c, has-miR-297, has-miR-573, has-let-7g, has-let-7f, hsa-miR-98, hsa-miR-758, hsa-let-7i, hsa-miR-34b, hsa-miR-583, hsa-miR-7, and has-miR-1.

IV. Nucleic Acids Methods

A. General Techniques

General texts which describe molecular biological techniques include Sambrook, Molecular Cloning: a Laboratory Manual (2.sup.nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, P. Tijssen, ed. Elsevier, N.Y. (1993); Berger and Kimmel, Guide to Molecular Cloning Techniques Methods in Enzymology volume 152 Academic Press, Inc., San Diego, Calif. These texts describe mutagenesis, the use of vectors, promoters and many other relevant topics related to, e.g., the generation and expression of genes that encode let-7 or any other miRNA activity. Techniques for isolation, purification and manipulation of nucleic acids, genes, such as generating libraries, subcloning into expression vectors, labeling probes, and DNA hybridization are also described in the texts above and are well known to one of ordinary skill in the art.

The nucleic acids, whether miRNA, DNA, cDNA, or genomic DNA, or a variant thereof, may be isolated from a variety of sources or may be synthesized in vitro. Nucleic acids as described herein can be administered to or expressed in humans, transgenic animals, transformed cells, in a transformed cell lysate, or in a partially purified or a substantially pure form.

Nucleic acids are detected and quantified in accordance with any of a number of general means well known to those of skill in the art. These include, for example, analytical biochemical methods such as spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, and the like, Southern analysis, Northern analysis, Dot-blot analysis, gel electrophoresis, RT-PCR, quantitative PCR, other nucleic acid or target or signal amplification methods, radiolabeling, scintillation counting, and affinity chromatography.

Various types of mutagenesis can be used, e.g., to modify a nucleic acid encoding a gene with miRNA activity. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, and mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like. Changes may be made to increase the activity of the miRNA, to increase its biological stability or half-life, and the like.

Comparative hybridization can be used to identify nucleic acids encoding genes with let-7 or other miRNA activity, including conservative variations of nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes part 1 chapter 2, “Overview of principles of hybridization and the strategy of nucleic acid probe assays,” (Elsevier, N.Y.), as well as in Ausubel, supra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

The term “stringent hybridization conditions” is meant to refer to conditions under which a nucleic acid will hybridize to its target subsequence, typically in a complex mixture of nucleic acid, but to no other sequences. Stringent conditions are sequence-dependent and will be different in different circumstances. Longer sequences hybridize specifically at higher temperatures. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5.times.SSC, and 1% SDS, incubating at 42° C. or, 5.times.SSC, 1% SDS, incubating at 65° C., with a wash in 0.2×SSC, and 0.1% SDS at 65° C.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

B. miRNA Forms

Suitable nucleic acids for use in the methods described herein include, but are not limited to, pri-miRNA, pre-miRNA, ds miRNA, mature miRNA or fragments of variants thereof that retain the biological activity of the miRNA and DNA encoding a pri-miRNA, pre-miRNA, mature miRNA, fragments or variants thereof, or DNA encoding regulatory elements of the miRNA.

miRNA can be modified in accordance with the invention emmpolying any suitable chemical moiety including, for example, phosphorothioate, boranophosphate, 2′-O-methyl, 2′-fluoro, PEG, terminal inverted-dT base, 2′tBDMS, or 2′-TOM or t′-ACE, LNA, and combinations thereof.

C. Vectors

In one embodiment, a nucleic acid encoding a miRNA molecule is on a vector is used as a source of the miRNA. These vectors include a sequence encoding a mature or hairpin (pri-miRNA, pre-miRNA, ds miRNA) miRNA and in vivo expression elements. In a preferred embodiment, these vectors include a sequence encoding a pre-miRNA and in vivo expression elements such that the pre-miRNA is expressed and processed in vivo into a mature miRNA. In another embodiment, these vectors include a sequence encoding the pre-miRNA gene and in vivo expression elements. In this embodiment, the primary transcript is first processed to produce the stem-loop precursor miRNA molecule. The stem-loop precursor is then processed to produce the mature microRNA.

Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

D. Promoters

The “in vivo expression elements” are any regulatory nucleotide sequence, such as a promoter sequence or promoter-enhancer combination, which facilitates the efficient expression of the nucleic acid to produce the microRNA. The in vivo expression element may, for example, be a mammalian or viral promoter, such as a constitutive or inducible promoter or a tissue specific promoter. Examples of which are well known to one of ordinary skill in the art. Constitutive mammalian promoters include, but are not limited to, polymerase promoters as well as the promoters for the following genes: hypoxanthine phosphoribosyl transferase (HPTR), adenosine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art. Particularly preferred promoters include those that activate transcription by RNA Polymerase II.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter.

Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter.

Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1).

Exemplary tissue-specific expression elements for gastric tissue include but are not limited to the human H+/K+-ATPase alpha subunit promoter.

Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter.

Exemplary tissue-specific expression elements for the endometrium include, but are not limited to, the uteroglobin promoter.

Exemplary tissue-specific expression elements for adrenal cells include, but are not limited to, cholesterol side-chain cleavage (SCC) promoter.

Exemplary tissue-specific expression elements for the general nervous system include, but are not limited to, gamma-gamma enolase (neuron-specific enolase, NSE) promoter.

Exemplary tissue-specific expression elements for the brain include, but are not limited to, the neurofilament heavy chain (NF-H) promoter.

Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p56lck) promoter, the humans CD2 promoter and its 3′ transcriptional enhancer, and the human NK and T cell specific activation (NKG5) promoter.

Exemplary tissue-specific expression elements for the colon include, but are not limited to, pp60c-src tyrosine kinase promoter, organ-specific neoantigens (OSNs) promoter, and colon specific antigen-P promoter.

Exemplary tissue-specific expression elements for breast cells include, but are not limited to, the human alpha-lactalbumin promoter.

Exemplary tissue-specific expression elements for the lung include, but are not limited to, the cystic fibrosis transmembrane conductance regulator (CFTR) gene promoter.

Other elements aiding specificity of expression in a tissue of interest can include secretion leader sequences, enhancers, nuclear localization signals, endosmolytic peptides, etc. Preferably, these elements are derived from the tissue of interest to aid specificity.

In general, the in vivo expression element shall include, as necessary, 5′ non-transcribing and 5′ non-translating sequences involved with the initiation of transcription. They optionally include enhancer sequences or upstream activator sequences.

E. Methods and Materials for Production of miRNA

The miRNA can be isolated from cells or tissues, recombinantly produced, or synthesized in vitro by a variety of techniques well known to one of ordinary skill in the art.

In one embodiment, miRNA is isolated from cells or tissues. Techniques for isolating miRNA from cells or tissues are well known to one of ordinary skill in the art. For example, miRNA can be isolated from total RNA using the mirVana miRNA isolation kit from Ambion, Inc. Another techniques utilizes the flashPAGE.TM. Fractionator System (Ambion, Inc.) for PAGE purification of small nucleic acids.

The miRNA can be obtained by preparing a recombinant version thereof (i.e., by using the techniques of genetic engineering to produce a recombinant nucleic acid which can then be isolated or purified by techniques well known to one of ordinary skill in the art). This embodiment involves growing a culture of host cells in a suitable culture medium, and purifying the miRNA from the cells or the culture in which the cells are grown. For example, the methods include a process for producing a miRNA in which a host cell containing a suitable expression vector that includes a nucleic acid encoding an miRNA is cultured under conditions that allow expression of the encoded miRNA. In a preferred embodiment the nucleic acid encodes let-7. The miRNA can be recovered from the culture, from the culture medium or from a lysate prepared from the host cells, and further purified. The host cell can be a higher eukaryotic host cell such as a mammalian cell, a lower eukaryotic host cell such as a yeast cell, or the host cell can be a prokaryotic cell such as a bacterial cell. Introduction of a vector containing the nucleic acid encoding the miRNA into the host cell can be effected by calcium phosphate transfection, DEAE, dextran mediated transfection, or electroporation (Davis, L. et al., Basic Methods in Molecular Biology (1986)).

Any suitable host/vector system can be used to express one or more of the miRNAs. These include, but are not limited to, eukaryotic hosts such as HeLa cells and yeast, as well as prokaryotic host such as E. coli and B. subtilis. miRNA can be expressed in mammalian cells, yeast, bacteria, or other cells where the miRNA gene is under the control of an appropriate promoter. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989). In the preferred embodiment, the miRNA is expressed in mammalian cells. Examples of mammalian expression systems include C127, monkey COS cells, Chinese Hamster Ovary (CHO) cells, human kidney 293 cells, human epidermal A43 1 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Mammalian expression vectors will comprise an origin of replication, a suitable promoter, polyadenylation site, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Potentially suitable yeast strains include Saccharomyces cerevisiae, Schizosaccharomyces pombe, Kluyveromyces strains, Candida, or any yeast strain capable of expressing miRNA. Potentially suitable bacterial strains include Escherichia coli, Bacillus subtilis, Salmonella typhimurium, or any bacterial strain capable of expressing miRNA.

In a preferred embodiment, genomic DNA encoding miRNA selected from the list of miRNAs: hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-miR-29a, hsa-miR-29a*, hsa-mir-29b-1, hsa-mir-29b-2, hsa-mir-29c, hsa-let-7g, hsa-miR-146b-5p, hsa-miR-154, hsa-miR-674 (mmu-miR-674), hsa-let-7f, hsa-miR-21, hsa-miR-22, hsa-miR-23a, hsa-miR-98, hsa-let-7a, hsa-miR-199a-3p, hsa-miR-214, hsa-miR-130a, hsa-miR-211, hsa-miR-515-5p, hsa-miR-517a, hsa-miR-517b, hsa-mir-203, hsa-miR-297, hsa-mir-573, hsa-mir-758, hsa-mir-583, hsa-mir-7, hsa-mir-1, is isolated, the miRNA is expressed in a mammalian expression system, RNA is purified and modified as necessary for administration to a patient. In a preferred embodiment the miRNA is in the form of a pre-miRNA, which can be modified as desired (i.e., for increased stability or cellular uptake).

Knowledge of DNA sequences of miRNA allows for modification of cells to permit or increase expression of an endogenous miRNA. Cells can be modified (e.g., by homologous recombination) to provide increased miRNA expression by replacing, in whole or in part, the naturally occurring promoter with all or part of a heterologous promoter so that the cells express the miRNA at higher levels. The heterologous promoter is inserted in such a manner that it is operatively linked to the desired miRNA encoding sequences. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also may be engineered to express an endogenous gene comprising the miRNA under the control of inducible regulatory elements, in which case the regulatory sequences of the endogenous gene may be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al.

The miRNA may be prepared by culturing transformed host cells under culture conditions suitable to express the miRNA. The resulting expressed miRNA may then be purified from such culture (i.e., from culture medium or cell extracts) using known purification processes, such as gel filtration and ion exchange chromatography. The purification of the miRNA may also include an affinity column containing agents which will bind to the protein; one or more column steps over such affinity resins as concanavalin A-agarose, heparin-toyopearl.TM. or Cibacrom blue 3GA Sepharose.TM.; one or more steps involving hydrophobic interaction chromatography using such resins as phenyl ether, butyl ether, or propyl ether; immunoaffinity chromatography, or complementary cDNA affinity chromatography.

The miRNA may also be expressed as a product of transgenic animals, which are characterized by somatic or germ cells containing a nucleotide sequence encoding the miRNA. A vector containing DNA encoding miRNA and appropriate regulatory elements can be inserted in the germ line of animals using homologous recombination (Capecchi, Science 244:1288-1292 (1989)), such that the express the miRNA. Transgenic animals, preferably non-human mammals, are produced using methods as described in U.S. Pat. No 5,489,743 to Robinson, et al., and PCT Publication No. WO 94/28122 by Ontario Cancer Institute. miRNA can be isolated from cells or tissue isolated from transgenic animals as discussed above.

In a preferred embodiment, the miRNA can be obtained synthetically, for example, by chemically synthesizing a nucleic acid by any method of synthesis known to the skilled artisan. The synthesized miRNA can then be purified by any method known in the art. Methods for chemical synthesis of nucleic acids include, but are not limited to, in vitro chemical synthesis using phosphotriester, phosphate or phosphoramidite cheminstry and solid phase techniques, or via deosynucleoside H-phosphonate intermediates (see U.S. Pat. No. 5,705,629 to Bhongle).

In some circumstances, for example, where increased nuclease stability is desired, nucleic acids having nucleic acid analogs and/or modified intemucleoside linkages may be preferred. Nucleic acids containing modified internucleoside linkages may also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH.sub.2—S—CH.sub.2), diinethylene-sulfoxide (—CH.sub.2—SO—CH.sub.2), dimethylene-sulfone (—CH.sub.2—SO.sub.2—CH.sub.2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., U.S. Pat. No. 5,714,606 to Acevedo, et al., U.S. Pat. No. 5,378,825 to Cook, et al., U.S. Pat. Nos. 5,672,697 and 5,466,786 to Buhr, et al., U.S. Pat. No. 5,777,092 to Cook, et al., U.S. Pat. No. 5,602,240 to De Mesmaeker, et al., U.S. Pat. No. 5,610,289 to Cook, et al. and U.S. Pat. No. 5,858,988 to Wang, also describe nucleic acid analogs for enhanced nuclease stability and cellular uptake.

V. Formulations

The compositions are administered to a patient in need of treatment or prophylaxis of at least one symptom or manifestation (since disease can occur/progress in the absence of symptoms) of cancer/proliferative disease. Aberrant expression of oncogenes is a hallmark of cancer. In a preferred embodiment, the cancer is lung cancer. In one embodiment, the compositions are administered in an effective amount to inhibit gene expression of one or more oncogenes. In preferred embodiments, the compositions are administered in an effective amount to inhibit gene expression of SPARC.

Methods for treatment or prevention of at least one symptom or manifestation of cancer are also described consisting of administration of an effective amount of a composition containing a nucleic acid molecule to alleviate at least one symptom or decrease at least one manifestation. In a preferred embodiment, the cancer is lung cancer. The compositions described herein can be administered in effective dosages alone or in combination with adjuvant cancer therapy such as surgery, chemotherapy, radiotherapy, thermotherapy, immunotherapy, hormone therapy and laser therapy, to provide a beneficial effect, e.g. reduce tumor size, reduce cell proliferation of the tumor, inhibit angiogenesis, inhibit metastasis, or otherwise improve at least one symptom or manifestation of the disease.

The nucleic acids described above are preferably employed for therapeutic uses in combination with a suitable pharmaceutical carrier. Such compositions comprise an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are described herein.

It is understood by one of ordinary skill in the art that nucleic acids administered in vivo are taken up and distributed to cells and tissues (Huang, et al., FEBS Lett. 558(1-3):69-73 (2004)). For example, Nyce et al. have shown that antisense oligodeoxynucleotides (ODNs) when inhaled bind to endogenous surfactant (a lipid produced by lung cells) and are taken up by lung cells without a need for additional carrier lipids (Nyce and Metzger, Nature, 385:721-725 (1997). Small nucleic acids are readily taken up into T24 bladder carcinoma tissue culture cells (Ma, et al., Antisense Nucleic Acid Drug Dev. 8:415-426 (1998). siRNAs have been used for therapeutic silencing of an endogenous genes by systemic administration (Soutschek, et al., Nature 432, 173-178 (2004)).

The nucleic acids described above may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The nucleic acids may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, thickeners and the like can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostatics, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative. The compositions may take such forms as.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The nucleic acids alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. For administration by inhalation, the nucleic acids are conveniently delivered in the form of an aerosol spray presentation from pressurized packs or a nebulizer, with the use of a suitable propellant.

In some embodiments, the nucleic acids described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the nucleic acids are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett. 14(19):4975-4977 (2004)) and in vivo (Soutschek, et al., Nature 432(7014):173-178 (2004)). In addition, it has been shown that binding of steroid conjugated oligonucleotides to different lipoproteins in the bloodstream, such as LDL, HDL, VLDL, or chylomicron, protect integrity and facilitate biodistribution (Rump, et al., Biochem. Pharmacol. 59(11):1407-1416 (2000)). Other groups that can be attached or conjugated to the nucleic acids described above to increase cellular uptake, include, but are not limited to, acridinederivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also described methods for enhanced delivery of nucleic acids molecules.

These pharmaceutical formulations may be manufactured in a manner that is itself known, e.g., by means of conventional mixing, dissolving, granulating, levigating, emulsifying, encapsulating, entrapping or lyophilizing processes.

The formulations described herein of the nucleic acids embrace fusions of the nucleic acids or modifications of the nucleic acids, wherein the nucleic acid is fused to another moiety or moieties, e.g., targeting moiety or another therapeutic agent. Such analogs may exhibit improved properties such as activity and/or stability. Examples of moieties which may be linked or unlinked to the nucleic acid include, for example, targeting moieties which provide for the delivery of nucleic acid to specific cells, e.g., antibodies to pancreatic cells, immune cells, lung cells or any other preferred cell type, as well as receptor and ligands expressed on the preferred cell type. Preferably, the moieties target cancer or tumor cells. For example, since cancer cells have increased consumption of glucose, the nucleic acids can be linked to glucose molecules. Monoclonal humanized antibodies that target cancer or tumor cells are preferred moieties and can be linked or unlinked to the nucleic acids. In the case of cancer therapeutics, the target antigen is typically a protein that is unique and/or essential to the tumor cells (e.g., the receptor protein HER-2).

VI. Methods of Treatment

A. Method of Administration

In general, methods of administering nucleic acids are well known in the art. In particular, the routes of administration already in use for nucleic acid therapeutics, along with formulations in current use, provide preferred routes of administration and formulation for the nucleic acids described above.

Nucleic acid compositions can be administered by a number of routes including, but not limited to: oral, intravenous, intraperitoneal, intramuscular, transdermal, subcutaneous, topical, sublingual, or rectal means. Nucleic acids can also be administered via liposomes. Such administration routes and appropriate formulations are generally known to those of skill in the art.

Administration of the formulations described herein may be accomplished by any acceptable method which allows the miRNA or nucleic acid encoding the miRNA to reach its target. The particular mode selected will depend of course, upon factors such as the particular formulation, the severity of the state of the subject being treated, and the dosage required for therapeutic efficacy. As generally used herein, an “effective amount” of a nucleic acids is that amount which is able to treat one or more symptoms of cancer or related disease, reverse the progression of one or more symptoms of cancer or related disease, halt the progression of one or more symptoms of cancer or related disease, or prevent the occurrence of one or more symptoms of cancer or related disease in a subject to whom the formulation is administered, as compared to a matched subject not receiving the compound or therapeutic agent. The actual effective amounts of drug can vary according to the specific drug or combination thereof being utilized, the particular composition formulated, the mode of administration, and the age, weight, condition of the patient, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intradermally for treatment or prevention of cancer, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

Preferably, the agent and/or nucleic acid delivery system are provided in a manner which enables tissue-specific uptake of the agent and/or nucleic acid delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the miRNA over a certain time period, for example, hours, days, weeks, months or years. This may be accomplished, for example, by repeated administrations of a formulation or by a sustained or controlled release delivery system in which the miRNA is delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include, but are not limited to, time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the miRNA is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the miRNA. In addition, a pump-based hardware delivery system may be used to deliver one or more embodiments.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the miRNA employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

Therapeutic compositions comprising one or more nucleic acids are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the nucleic acids at various concentrations, e.g., as applied to the mass and overall health of the patient. Administration can be accomplished via single or divided doses.

In vitro models can be used to determine the effective doses of the nucleic acids as a potential cancer treatment. Suitable in vitro models include, but are not limited to, proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella et al., J. Natl. Can. Inst., 52: 921-30 (1974), mobility and invasive potential of tumor cells in Boyden Chamber assays as described in Pilkington et al., Anticancer Res., 17: 4107-9 (1997), and angiogenesis assays such as induction of vascularization of the chick chorioallantoic membrane or induction of vascular endothelial cell migration as described in Ribatta et al., Intl. J. Dev. Biol., 40: 1189-97 (1999) and Li et al., Clin. Exp. Metastasis, 17:423-9 (1999), respectively. Suitable tumor cells lines are available, e.g. from American Type Tissue Culture Collection catalogs.

In vivo models are the preferred models to determine the effective doses of nucleic acids described above as potential cancer treatments. Suitable in vivo models include, but are not limited to, mice that carry a mutation in the KRAS oncogene (Lox-Stop-Lox K-Ras.sup.G12D mutants, Kras2.sup.tm4TYj) available from the National Cancer Institute (NCI) Frederick Mouse Repository. Other mouse models known in the art and that are available include but are not limited to models for gastrointestinal cancer, hematopoietic cancer, lung cancer, mammary gland cancer, nervous system cancer, ovarian cancer, prostate cancer, skin cancer, cervical cancer, oral cancer, and sarcoma cancer.

In determining the effective amount of the miRNA to be administered in the treatment or prophylaxis of disease the physician evaluates circulating plasma levels, formulation toxicities, and progression of the disease.

The dose administered to a 70 kilogram patient is typically in the range equivalent to dosages of currently-used therapeutic antisense oligonucleotides such as Vitravene.RTM. (fomivirsen sodium injection) which is approved by the FDA for treatment of cytomegaloviral RNA, adjusted for the altered activity or serum half-life of the relevant composition.

The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, the nucleic acids can also be administered in therapeutically effective amounts as a portion of an anti-cancer cocktail. An anti-cancer cocktail is a mixture of the oligonucleotide or modulator with one or more anti-cancer drugs in addition to a pharmaceutically acceptable carrier for delivery. The use of anti-cancer cocktails as a cancer treatment is routine. Anti-cancer drugs that are well known in the art and can be used as a treatment in combination with the nucleic acids described herein include, but are not limited to: Actinomycin D, Aminoglutethimide, Asparaginase, Bleomycin, Busulfan, Carboplatin, Carmustine, Chlorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Floxuridine, 5-Fluorouracil (5-Fu), Flutamide, Hydroxyurea (hydroxycarbamide), Ifosfamide, Interferon Alpha-2a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Octreotide, Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate.

B. Diseases for Treatment with the Invention

Proliferative diseases, e.g., from the group consisting of hypertrophic scars and keloids, proliferative diabetic retinopathy, rheumatoid arthritis, arteriovenous malformations, atherosclerotic plaques, delayed wound healing, hemophilic joints, nonunion fractures, Osler-Weber syndrome, psoriasis, pyogenic granuloma, scleroderma, tracoma, menorrhagia, vascular adhesions and restenosis.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness. Therapeutic formulations described herein may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma. Therapeutic formulations can be administered in therapeutically effective dosages alone or in combination with adjuvant cancer therapy such as surgery, chemotherapy, radiotherapy, thermotherapy, immunotherapy, hormone therapy and laser therapy, to provide a beneficial effect, e.g. reducing tumor size, slowing rate of tumor growth, reducing cell proliferation of the tumor, promoting cancer cell death, inhibiting angiongenesis, inhibiting metastasis, or otherwise improving overall clinical condition, without necessarily eradicating the cancer.

Cancers include, e.g., biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. In a preferred embodiment, the formulations are administered for treatment or prevention of lung cancer.

In addition, therapeutic nucleic acids may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the nucleic acids to reduce the risk of developing cancers. In one embodiment, a nucleic acid in a suitable formulation may be administered to a subject who has a family history of cancer, or to a subject who has a genetic predisposition for cancer. In other embodiments, the nucleic acid in a suitable formulation is administered to a subject who has reached a particular age, or to a subject more likely to get cancer. In yet other embodiments, the nucleic acid in a suitable formulation is administered to subjects who exhibit symptoms of cancer (e.g., early or advanced). In still other embodiments, the nucleic acid in a suitable formulation may be administered to a subject as a preventive measure. In some embodiments, the nucleic acid in a suitable formulation may be administered to a subject based on demographics or epidemiological studies, or to a subject in a particular field or career.

The following examples further illustrate the invention but, of course, should not be construed as in any way limiting its scope.

### EXAMPLE 1

This example demonstrates the differential expression of an exogenous SPARC gene in different cell lines.

The cloned full length cDNA for SPARC (containing a Q3 mutation, see United States Patent Appl. Pub. No. 20070117133, issued as U.S. Pat. No. 7,332,568) was subcloned into an expression plasmid (pVT1000Q3) behind the CMV promoter. An epitope tag with six histidine residues was engineered into the carboxyl terminus of the protein by primer directed mutagenesis- giving rise to “SPARC-6His”. The SPARC-6His plasmid was transfected into CHO and 293 cells. Cell and conditioned media were evaluated for SPARC expression by separation on SDS-PAGE, transferred to nitrocellulose membrane, and immunoblotted using a monoclonal anti-His-tag antibody (Qiagen, Calif., USA) and alkaline phosphatase-conjugated secondary antibody (Pierce Biotechnology Inc, Ill., USA). The bands were developed by 1-step NBT/BCIP kit (Pierce Biotechnology Inc, Ill., USA).

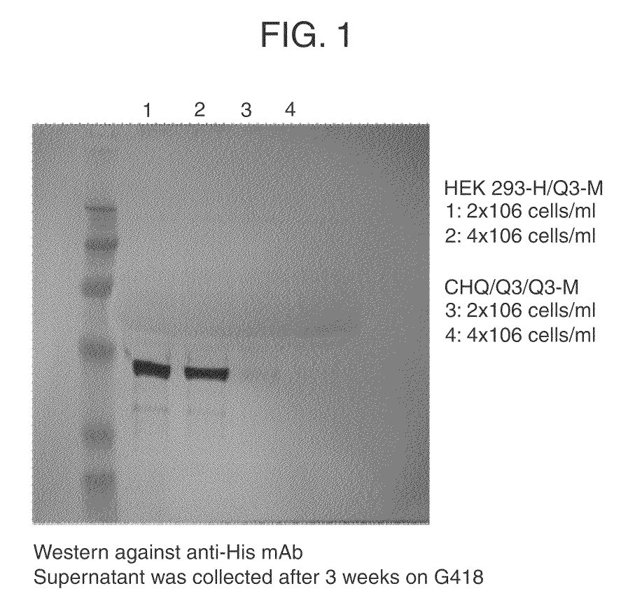

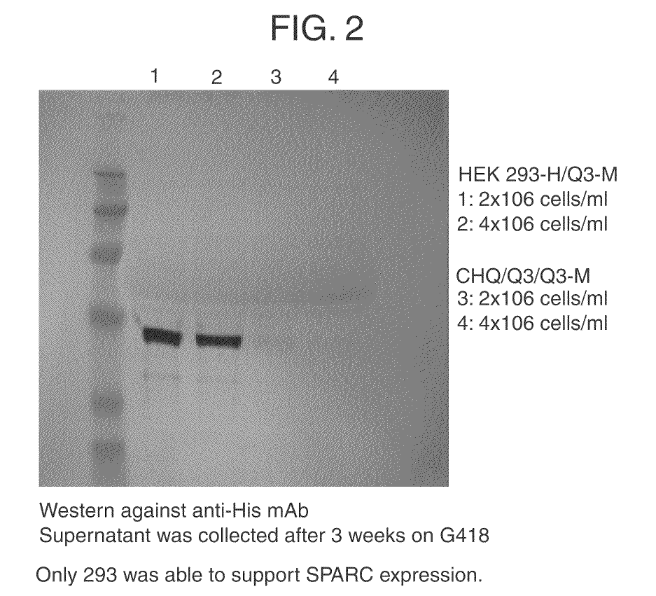

As shown in FIGS. 1 and 2, at 48 hours and 3 weeks post-transfection, respectively, there was no expression of SPARC-6His in CHO cells even though the 293 transfectants demonstrated the construct was competent.

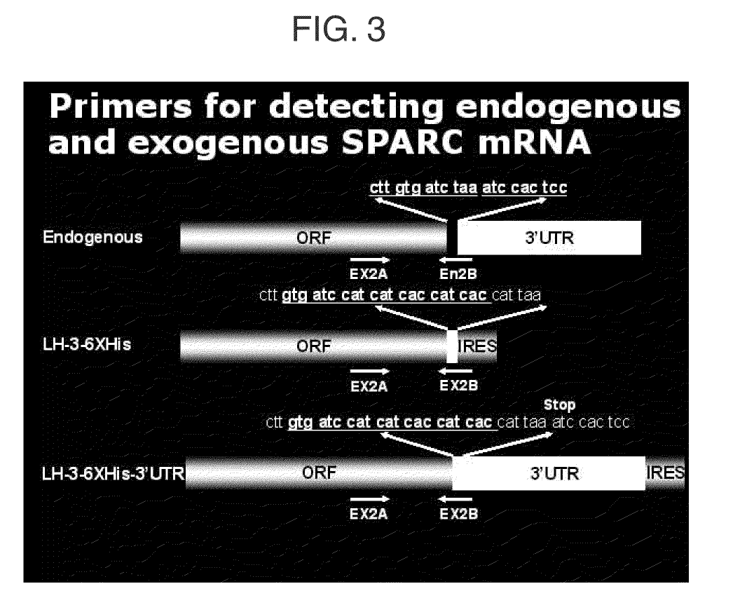

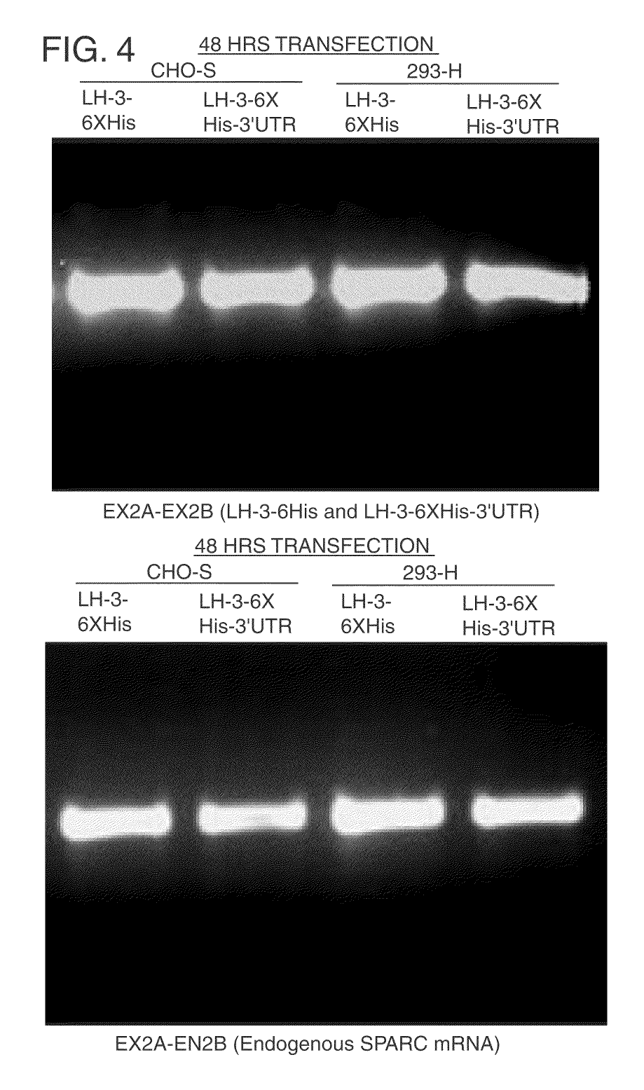

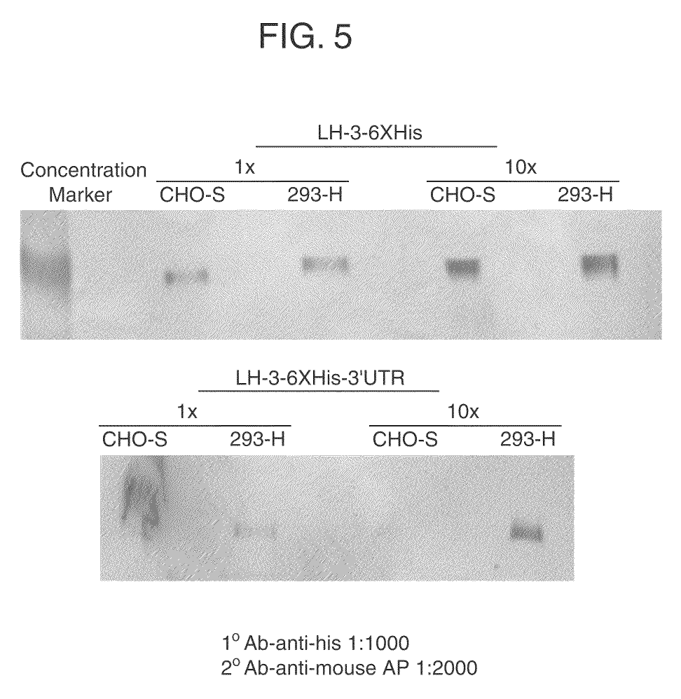

To consider whether the 3′UTR is involved inhibiting expression in CHO cells two constructs were made: 1) LH-3-6×His which has only the coding region of SPARC cDNA and its 5′UTR and 2) LH-3-6×His-3′UTR which has the full length cDNA and therefore the 3′UTR. Schematics of these constructs are shown in FIG. 3. Both constructs are driven by the CMV promoter. The plasmids were transfected into CHO or 293 cells using Lipofectamine 2000 (Invitrogen, Calif., USA). At 48 hrs post-transfection, cells were collected and total RNA was isolated with RNeasy Mini kit (Qiagen, Calif., USA). RT-PCR was performed to confirm the presence of endogenous and exogenous SPARC mRNAs; the location of these primers are shown in FIG. 3. The endogenous SPARC mRNA expression level was detected with two primers (EX2A: 5′-GGGTGAAGAAGATCCATGAG-3′) and (EN2B: 5′-GGAGTGGATTTAGATCACAAG-3′). The exogenous SPARC mRNA expression level was detected with two primers (EX2A: 5′-GGGTGAAGAAGATCCATGAG′-3′) and (EX2B: 5′-GTGATGGTGATGATG GATCAC-3′). As shown in FIG. 4, both endogenous (derived from chromosomal SPARC gene) and exogenous (derived from transfected plasmids) SPARC mRNAs were detected in CHO and 293 at 48 hr. In addition, at 48 hours post-transfection, the condition medium was subjected to Western blot analysis (FIG. 5). (In order to visualize the protein, the samples were concentrated 10× and ran along with the unconcentrated 1× materials.) Surprisingly, as shown in FIG. 5, SPARC protein was detected from CHO cells transformed with the LH-3-6×His construct, but not the LH-3-6×His-3′UTR construct. In contrast, when 293 cells were transfected, expressed protein encoded by either construct was detected. These result is consistent with the 3′UTR being responsible for translational inhibition of SPARC expression in CHO and with a the presence of different expression environments in CHO and 293 cells.

### EXAMPLE 2

This example demonstrates a method of readily confirming suppression of translation involving the SPARC 3′UTR using luciferase report gene.

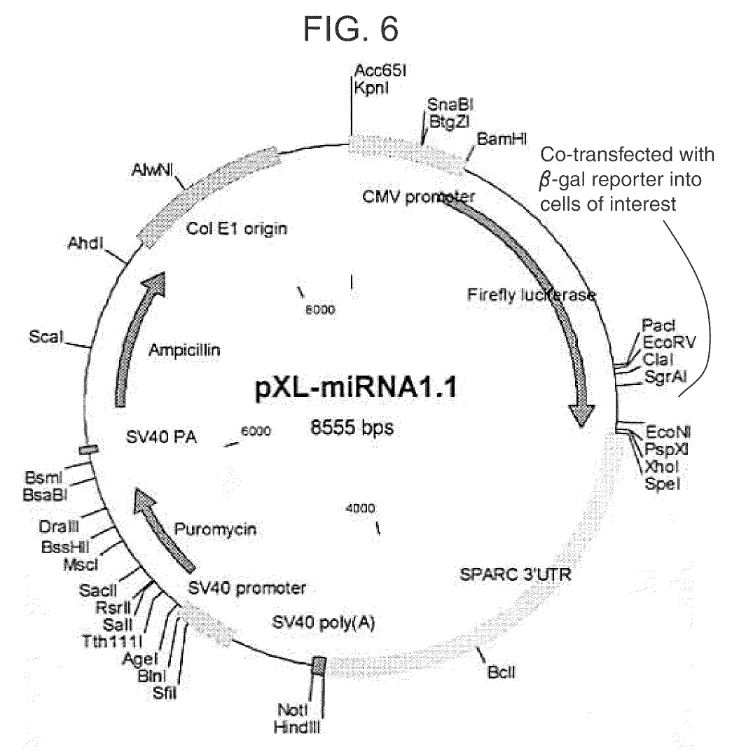

The SPARC's 3′UTR encoding region was moved into the pMIR reporter plasmid (Ambion, Tex.) behind the luciferase coding region to generate pXL-miRNA1.1. The physical map of the plasmid is shown in FIG. 6. 20,000-30,000 cells were seeded in 96-well plates and transfected with 170 ng of pMIR-reporter or pXL-miRNA1.1 and 30 ng of pMIR-reporter-β-gal control vector (Ambion, Tex.) using Lipofectamine 2000 (Invitrogen, Calif.). Cell lysate was collected and assayed 24 or 48 hours post-transfection. Firefly and β-galactosidase activities were measured using a Dual-Light Luciferase and β-Galactosidase Reporter Gene Assay System (Applied Biosystems, Calif.) according to the manufacture's protocol. The luciferase activity was normalized with β-galactosidase activity. The relative luciferase activity was expressed as the ratio of the normalized luciferase activity with 3′UTR to that without 3′UTR.

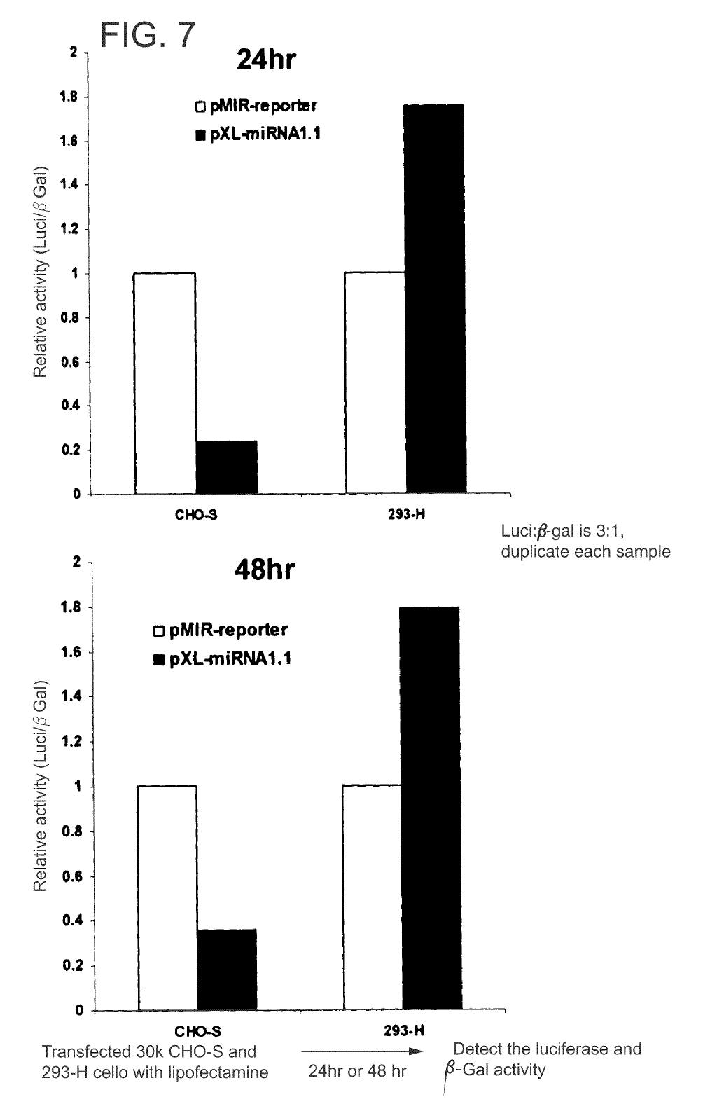

As shown in FIG. 7, the presence of SPARC 3′UTR repressed luciferase expression significantly in CHO cells. This suppression was not observed in 293 cells. This is consistent with the data shown in Example 1 for SPARC. Therefore, the SPARC 3′UTR contains element(s) which cause translational suppression in CHO cells. The activity is consistent with that mediated by a miRNA.

To confirm that SPARC's 3′UTR translational inhibition is not unique to CHO, we performed the luciferase transfection experiment with a pMIR- reporter, pXL-miRNA1.1 and pXL-miRNA1.2. The two plasmid pXL-miRNA1.1 and pXL-miRNA1.2 both have the SPARC's 3′UTR behind the coding region of luciferase and are different from one another by a small 14 bp deletion at the coding region 3′UTR junction in pXL-miRNA1.1.

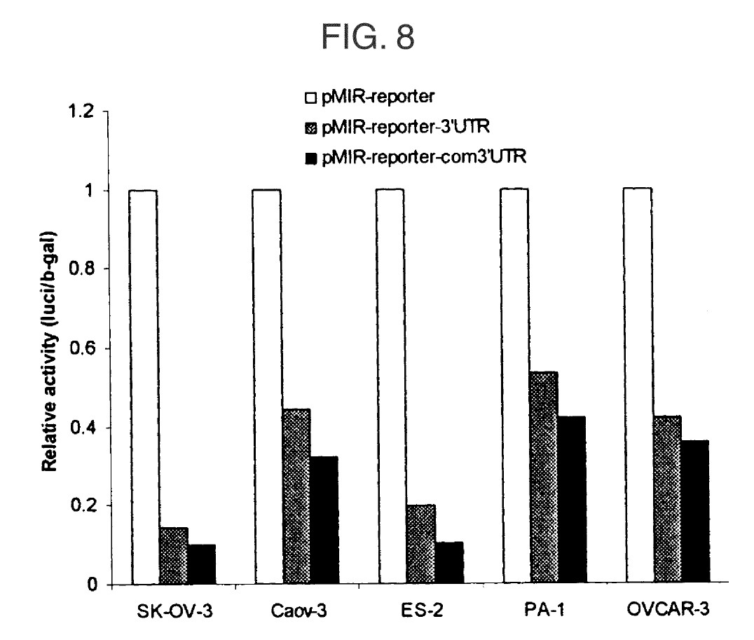

Transfection into ovarian cell lines shown that SPARC's 3′UTR translation inhibition was effective among all these cell lines. FIG. 8 shows the inhibition of luciferase translation by SPARC's 3′UTR from either the pXL-miRNA1.1 or pXL-miRNA1.2 constructs in SK-OV-3, Caov-3, ES-2, PA-1, and OVCAR-3 cells.

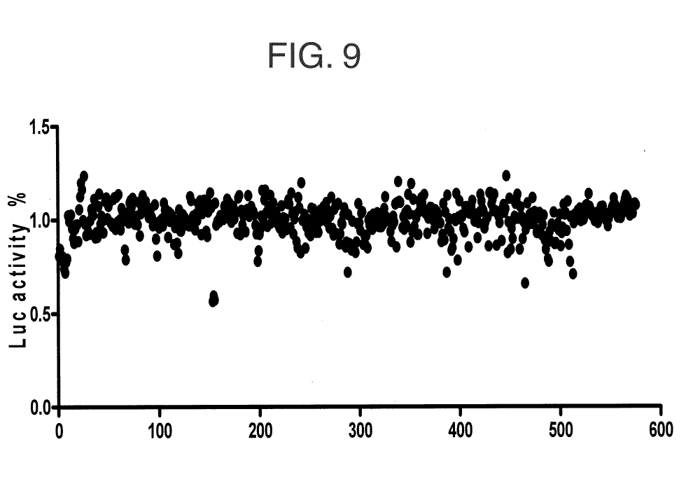

A pre-miRNA library (Ambion, Austin, Tex.) was screen using the Luciferase-SPARC 3′UTR reporter system in 293 cells. This identified four miRNAs that inhibited luciferase: hsa-mir-29a, hsa-mir-29b, hsa-mir-29c, hsa-miR-297, hsa-mir-573, hsa-mir-758, hsa-mir-583, hsa-mir-7, hsa-mir-1 (FIG. 9 outliers).

### EXAMPLE 3

This Example demonstrates the presence of differentially expressed endogenous miRNAs in CHO, which exhibits miRNA mediated suppression of SPARC expression, and 293 which does not.

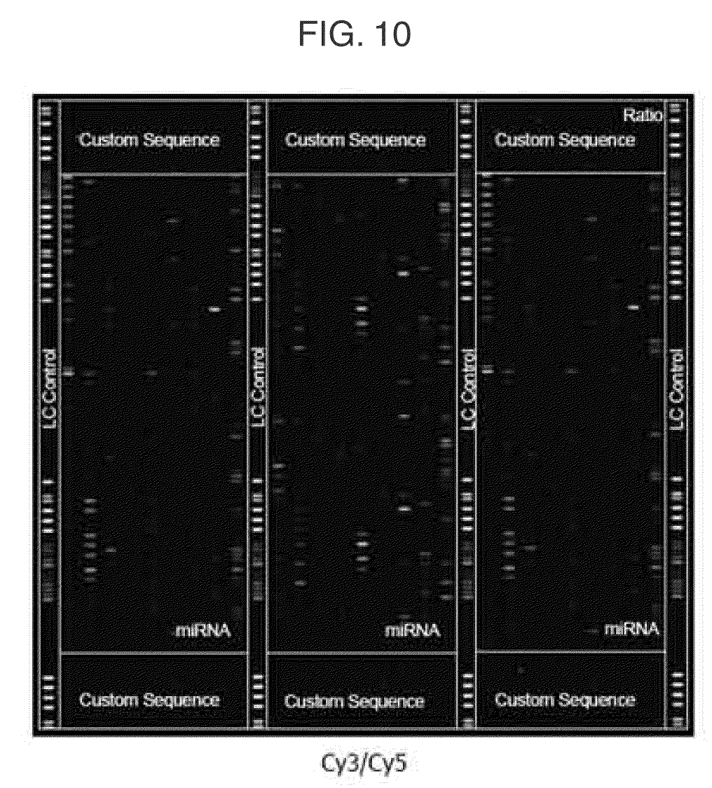

To identify the miRNA(s) involved in the suppression of SPARC in CHO cells, purified miRNA from CHO and 293 cells and labeled them with Cy3 and Cy5, respectively. The labeled miRNAs were simultaneously hybridized to a panel of miRNA complementary sequence in a microarray format. The data are shown in FIG. 10.

Quantitative analysis revealed s series of human miRNA was identified which exhibited upregulation in CHO and absences or low levels in 293 cells (ratio being greater than 10). The identified CHO miRNAs are prime candidates for use as SPARC suppressing miRNAs and include: hsa-miR-885-5p, hsa-let-7b, hsa-let-7i, hsa-miR-186, hsa-miR-125b, hsa-let-7d, hsa-miR-34c-5p, hsa-miR-139-5p, hsa-miR-100, hsa-miR-34b, hsa-let-7c, hsa-let-7d, hsa-miR-29a, hsa-let-7g, hsa-miR-146b-5p, hsa-miR-154, hsa-miR-674, hsa-let-7f, hsa-miR-21, hsa-miR-22, hsa-miR-23a, hsa-miR-98, hsa-let-7a, hsa-miR-199a-3p, hsa-miR-214, and hsa-miR-130a.

Computational analysis of the 3′UTR of SPARC also revealed a series of potential miRNAs which would target SPARC 3′UTR. These miRNAs are hsa-miR-211, hsa-miR-515-5p, hsa-miR-517a, hsa-miR-517b, hsa-mir-29, and hsa-mir-203.1

### EXAMPLE 4

This example demonstrates the ability of forced SPARC expression to alter gene expression of both murine and human genes. Forced expression is equivalent to the use of exogenous miRNAs to counteract the effect of endogenous SPARC-inhibitory miRNAs. In the systems disclosed in this Example, untransfected cells expression pattern is equivalent to the result produced by the administration of or expression of SPARC inhibitory miRNAs.

Two cell lines, PC3 (human prostate cancer) and HT29 (human colon cancer), were engineered to express exogenous SPARC. The SPARC expressing tumor cells were studied in human-mouse xenograft models systems. Microarray analysis was used to detect changes in gene expression induced by SPARC expression or inhibition in both tumor and stromal cells. The xenograft results were compared to control xenografts from untransfected HT29 or PC3 cells.

The following table list the Genbank and miRNa accession numbers for transcripts modulated by SPARC expression in both models:

| Mouse mRNA | Human mRNA | Mouse miRNA | Human |
|---|---|---|---|
| (2-4 fold | (2-13 fold | (20-30 fold | miRNA (14-100 |
| modulation) | modulation) | induction) | fold inhibition) |
| NM_133664, | NM_016619, | rno-miR-377, | hsa-miR-542-5p, |
| NM_011641, | NM_016323, | (mmu-mir-377) | hsa-miR-186 |
| NM_010226, | NM_012294, | | |
| NM_008380, | NM_006393, | | |
| BB480262, | NM_005609, | | |
| AW909062 | NM_002462, | | |
| | NM_002346, | | |
| | NM_001955, | | |
| | NM_001548, | | |
| | NM_000909, | | |
| | BM930167, | | |
| | BM874773, | | |
| | BI560717, | | |
| | AW511255, | | |
| | AK098543, | | |
| | AI860360, | | |
| | AI760944 | | |

The human genes up-regulated by 2 fold or more by the expression of SPARC in the HT29 system were:

### Fold Change Gene

3.71 neuropeptide Y receptor Y1
2.54 phosphorylase, glycogen; muscle (McArdle syndrome, glycogen storage disease type V)
2.13 lymphocyte antigen 6 complex, locus E
2.47 interferon-induced protein with tetratricopeptide repeats 1
2.55 hect domain and RLD 5
2.11 placenta-specific 8
2.08 myxovirus (influenza virus) resistance 1, interferon-inducible protein p78 (mouse)
2.10 ATPase, H+ transporting, lysosomal 56/58 kDa, V1 subunit B, isoform 2

The human genes down-regulated by 2 fold or more by the expression of SPARC in the HT29 system were:

### Fold Change Gene

0.47 endothelin 1
0.48 Tropomyosin 1 (alpha)
0.46 nebulette
0.49 Rap guanine nucleotide exchange factor (GEF) 5
0.34 Similar to CG14853-PB
0.43 Microtubule associated monoxygenase, calponin and LIM domain containing
0.22 Transcribed locus, weakly similar to XP_{—}517655.1 PREDICTED: similar to KIAA0825 protein [Pan troglodytes]
0.27 Forkhead box P1
0.27 Phosphodiesterase 10A

The human genes up-regulated by 2 fold or more by the expression of SPARC in the PC3 system were:

### Fold Change Gene

3.71 neuropeptide Y receptor Y1
5.32 Complement factor B
4.49 Stathmin 1/oncoprotein 18
2.67 Myxovirus (influenza virus) resistance 1, interferon-inducible protein p78 (mouse)
2.55 hect domain and RLD 5
2.90 Interleukin-1 receptor-associated kinase 4
2.69 Leucine rich repeat containing 25
2.12 ATPase, H+ transporting, lysosomal 56/58 kDa, V1 subunit B2

The human genes down-regulated by 2 fold or more by the expression of SPARC in the PC3 system were:

### Fold Change Gene

0.36 Endothelin 1
0.32 Tropomyosin 1 (alpha)
0.28 Coagulation factor V (proaccelerin, labile factor)
0.24 Rap guanine nucleotide exchange factor (GEF) 5
0.36 Similar to CG14853-PB
0.25 Transient receptor potential cation channel, subfamily M, member 2
0.42 RGM domain family, member B
0.31 Forkhead box P1
0.26 Kelch domain containing 5

The murine genes showing a 2 fold or greater up- or down-regulation by the expression of SPARC in the HT29 system were:

### Fold Change Gene

4.33 transformation related protein 63
2.81 PREDICTED: hypothetical protein XP_143616 [Mus musculus], mRNA sequence sequence
2.60 PREDICTED: similar to DEP domain containing 2 isoform a [Mus musculus], mRNA sequence
2.26 ladinin
forkhead-like 18 (Drosophila)
0.27 inhibin beta-A

The murine genes showing a 2 fold or greater up- or down-regulation by the expression of SPARC in the PC3 system were:

### Fold Change Gene

3.30 transformation related protein 63
2.36 PREDICTED: hypothetical protein XP_{—}143616 [Mus musculus], mRNA sequence
2.21 PREDICTED: similar to DEP domain containing 2 isoform a [Mus musculus], mRNA sequence
2.42 ladinin
0.50 forkhead-like 18 (Drosophila)
0.46 inhibin beta-A

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for inhibiting the expression of SPARC protein in the cells of an organism comprising administering to the organisms an inhibitorily effective amount of one or more miRNAs that bind to endogenous SPARC RNA and inhibit SPARC protein expression in the cells of the organism.

2. The method of claim 1 wherein the miRNA is selected from the group consisting of a pri-miRNA, pre-miRNA, ds miRNA, mature miRNA, and fragments or variants thereof.

3. The method of claim 2, wherein the miRNA is from 10 nucleotides to 170 nucleotides in length and reduces reporter activity expressed from a construct that encodes a transcript for the reporter fused to the SPARC mRNA 3′ untranslated region.

4. The method of claim 3, wherein the miRNA is from 10 to 50 nucleotides in length.

5. The method of claim 2, wherein the miRNA target sequence is SPARC and the miRNA hybridizes under stringent conditions to the complementary sequence of any one or more sequences selected from the group consisting of SEQ ID NOS: 1-41 and 44-83.

6. The method of claim 2, wherein the miRNA target sequence is SPARC and the miRNA has at least 90% sequence identity to one or more of SEQ ID NOS: 1-41 and 44-83.

7. The method of claims 3, wherein the miRNA is a synthetic RNA or is encoded by an isolated nucleic acid.

8. The method of claim 3, wherein the isolated nucleic acid further comprises a vector.

9. The method of claim 8, wherein the vector is selected from the group consisting of a plasmid, cosmid, phagemid, virus, and artificial chromosome.

10. The method of claim 8, wherein the vector further comprises one or more in vivo expression control elements.

11. The method of claim 10, wherein the in vivo expression control element is selected from the group consisting of a promoter, enhancer, RNA splicing signal, and combinations thereof.

12. The method of any one of claims 7, wherein the isolated nucleic acid is transfected into the cells of the organism.

13. The method of claim 7, wherein the miRNA is synthetic and administered as a naked RNA.

14. The method of claim 7, wherein the miRNA is synthetic and administered as a chemically modified RNA.

15. The method of claim 14, wherein the synthetic miRNA is modified with a chemical moiety selected from the group consisting of phosphorothioate, boranophosphate, 2′-O-methyl, 2′-fluoro, PEG, terminal inverted-dT base, 2′tBDMS, 2′-TOM, t′-ACE, LNA (locked nucleic acid), and combinations thereof.

16. The method of claim 7, wherein the miRNA is synthetic and administered in a liposome, polymer-based nanoparticle, cholesterol conjugate, cyclodextran complex, polyethylenimine polymer or a protein complex, or as naked miRNA, naked DNA, naked LNA or as complex with RISC.

17. The method of claim 7, wherein the miRNA is synthetic and is administered directly to the diseased tissue, intravenously, subcutaneously, intramuscularly, nasally, intraperitonealy, vaginally, anally, orally, intraocularly or intrathecally.

18. The method of claim 1, wherein the miRNA is administered to an organism afflicted with cancer, restenosis, other proliferative disease, osteoporosis or wound healing.

19. The method of claim 18, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the other proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas.

20. The method of claim 19, wherein the organism is a human undergoing one or more cancer therapies selected from the group consisting of surgery, chemotherapy, radiotherapy, thermotherapy, immunotherapy, hormone therapy and laser therapy.

21. The method of claim 19, wherein organism is a human undergoing one or more antiproliferative therapies consisting of surgery, chemotherapy, radiotherapy, thermotherapy, immunotherapy, hormone therapy, laser therapy, or stenting.

22. The method of claim 19 wherein the organism is a human.

23. A method of inhibiting the expression of one or more proteins in the cells of an organism, wherein the proteins are selected from the group consisting of clusterin, β chain; clusterin, α chain; N-cadherin; secemin 1; collagen, type v, α-chain; renin, βchain; renin; and cytokeratin I, type II, and wherein the method comprises administering to the organism an inhibitorily effective amount of one or more miRNAs that bind to and inhibit SPARC expression in the cells of the organism.

24. A method of increasing the expression of one or more proteins in the cells of an organism, wherein the proteins are selected from the group consisting of α-actin; hsp27; collagen, type I, α-2 chain; peroxiredoxin 3; β-5 tubulin; p32, chain and wherein the method comprising comprises administering to the organism an inhibitorily effective amount of one or more miRNAs that bind to and inhibit SPARC expression in the cells of the organism.

25. A method of modulating the expression of one or more proteins in the cells of an organism, wherein the proteins are selected from the group consisting of
(a) the following Genebank accession numbers: NM_{—}016619, NM_{—}016323, NM_{—}012294,NM_{—}006393, NM_{—}005609, NM_{—}002462, NM_{—}002346, NM_{—}001955, NM_{—}001548, NM_{—}000909, BM930167, BM874773, B1560717, AW511255, AK098543, A1860360, A1760944;
(b) human counterpart of the following mouse mRNAs: NM_{—}133664, NM_{—}011641, NM_{—}010226, NM_{—}008380, BB480262, AW909062;
(c) human miRNAs: hsa-miR-542-5p, hsa-miR-186; and
(d) human counterparts of the following mouse miRNAs: mo-miR-377, mmu-mir-377;
said method comprising administering to the organism an inhibitorily effective amount of one or more miRNAs that bind to and inhibit SPARC expression in the cells of the organism.

26. A therapeutic composition for administration to a patient in need of therapy for cancer, restenosis, other proliferative diseases, osteoporosis or wound healing, comprising an isolated nucleic acid for the expression in the cells of the patient of an effective amount of miRNA to bind to SPARC mRNA and inhibit expression of SPARC protein.

27. The therapeutic composition of claim 26, wherein the miRNA is selected from the group consisting of a pri-miRNA, pre-miRNA, ds miRNA, mature miRNA, and fragments or variants thereof.

28. The therapeutic composition of claim 27, wherein the isolated nucleic acid is a vector selected from the group consisting of a plasmid, cosmid, phagemid, virus, and artifical chromosome.

29. The therapeutic composition of claim 28, wherein the isolated nucleic acid further comprises one or more in vivo expression control elements selected from the group consisting of a promoter, enhancer, RNA splicing signal, and combinations thereof.

30. The therapeutic composition of claim 26, wherein the miRNA is synthetic and administered as a naked RNA.

31. The therapeutic composition of claim 26, wherein the miRNA is synthetic and administered as a chemically modified RNA.

32. The therapeutic composition of claim 31, wherein the synthetic miRNA is modified with a chemical moiety selected from the group consisting of phosphorothioate, boranophosphate, 2′-O-methyl, 2′-fluoro, terminal inverted-dT bases, PEG, 2′tBDMS, 2′-TOM, t′-ACE, LNA, and combinations thereof.

33. The therapeutic composition of claim 26, wherein the miRNA is synthetic and administered in a liposome, polymer-based nanoparticle, cholesterol conjugate, cyclodextran complex, polyethylenimine polymer or a protein complex, or as naked miRNA or as complex with RISC.

34. The therapeutic composition of claim 26, wherein the miRNA is synthetic and is administered directly to the diseased tissue, intravenously, subcutaneously, intramuscularly, nasally, intraperitonealy, vagainally, anally, orally, intraocularly or intrathecally.

35. The therapeutic composition of claim 34, wherein the miRNA is from 10 nucleotides to 170 nucleotides in length and reduces reporter activity expressed from a construct that encodes a transcript for the reporter fused to the SPARC mRNA 3′ untranslated region.

36. The therapeutic composition of claim 35, wherein the miRNA is from 10 to 50 nucleotides in length.

37. The therapeutic composition of claim 26, wherein the miRNA target sequence is SPARC and the miRNA hybridizes under stringent conditions to the complementary sequence of any one or more sequences selected from the group consisting of SEQ ID NOS: 1-41 and 44-83.

38. The therapeutic composition of claim 26, wherein the miRNA has at least 90% identity to one or more of the sequences in the group consisting of: SEQ ID NOS: 1-41 and 44-83.

39. The therapuetic composition of claim 26, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, comary artery disease, delayed wound healing, hemophilic joints, nonunion fractures, Osler-Weber syndrome, psoriasis, pyogenic granuloma, scleroderma, tracoma, menorrhagia, vascular adhesions, and papillomas.

40. A method for increasing the expression of SPARC protein in the cells of an organism comprising administering to the organism an effective amount of one or more antagonistic miRNAs that bind to one or more endogenous miRNAs and reverse the inhibition of SPARC protein expression caused by the endogenous miRNA.

41. The method of claim 40, wherein the antagonistic miRNA is from 10 nucleotides to 170 nucleotides in length and induces reporter activity expressed from a construct that encodes a transcript for the reporter fused to the SPARC mRNA 3′ untranslated region.

42. The method of claim 41, wherein the antagonistic miRNA is from 10 to 50 nucleotides in length.

43. The method of claim 41, wherein the antagonistic miRNA has a nucleic acid sequence that is at least 90% complementary to a sequence from the group consisting of: SEQ ID NOS: 1-41 and 44-83 and combinations thereof, wherein thymidine and uracil are treated as the same nucleotide.

44. The method of claim 41, wherein the antagonistic miRNA is a synthetic nucleic acid, or is encoded by an isolated nucleic acid.

45. The method of claim 44, wherein the isolated nucleic acid comprises a vector.

46. The method of claim 45, wherein the vector is selected from the group consisting of a plasmid, cosmid, phagemid, virus, and artificial chromosome.

47. The method of claim 46, wherein the vector further comprises one or more in vivo expression control elements.

48. The method of claim 47, wherein the in vivo expression control element is selected from the group consisting of a promoter, enhancer, RNA splicing signal, and combinations thereof.

49. The method of claim 44, wherein the isolated nucleic acid is transfected into the cells of the organism.

50. The method of claim 44, wherein the antagonistic miRNA is synthetic and administered as a naked nucleic acid.

51. The method of claim 44, wherein the antagonistic miRNA is synthetic and administered as a chemically modified nucleic acid.

52. The method of claim 51, wherein the synthetic antagonistic miRNA is modified with a chemical moiety selected from the group consisting of phosphorothioate, boranophosphate, 2′-O-methyl, 2′-fluoro, terminal inverted-dT bases, PEG, 2′tBDMS, or 2′-TOM, t′-ACE, LNA, and combinations thereof.

53. The method of claim 44, wherein the antagonistic miRNA is synthetic and administered in a lipoprotein complex, liposome, polymer-based nanoparticle, cholesterol conjugate, cyclodextran complex, polyethylenimine polymer or a protein complex, or as naked DNA, naked RNA or a LNA.

54. The method of claim 44, wherein the antagonistic miRNA is synthetic and is administered to the diseased tissue in the organism, intravenously, subcutaneously, intramuscularly, nasally, intraperitonealy, vagainally, anally, orally, intraocularly or intrathecally.

55. The method of claim 44, wherein the organism is a human patient and the antagonist is administered to the patient for treatment or prevention of cancer, restenosis or other proliferative diseases, osteoporosis or exaggerated wound healing.

56. The method of claim 55, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the other proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas.

57. The method of claim 56, wherein the organism is a human patient is undergoing one or more cancer therapies selected from the group consisting of surgery, chemotherapy, radiotherapy, thermotherapy, immunotherapy, hormone therapy and laser therapy.

58. The method of claim 56, wherein the organism is a human patient is undergoing one or more antiproliferative therapies consisting of surgery, chemotherapy, radiotherapy, thermotherapy, immunotherapy, hormone therapy, laser therapy, or stenting.

59. The method of claim 56, wherein the organism is a human.

60. A method of increasing the expression of one or more proteins in the cells of an organism, the protein being selected from the group consisting of clusterin, β chain; clusterin, α chain; N-cadherin; secernin 1; collagen, type v, α-chain; renin, βchain; renin; and cytokeratin I, type II comprising administering to the organism an effective amount of one or more antagonistic miRNAs that bind to one or more endogenous miRNAs so as to reverse the inhibition of SPARC protein expression by the endogenous miRNA.

61. A method of decreasing the expression of one or more proteins in the cells of an organism, the protein being selected from the group consisting of α-actin; hsp27; collagen, type I, α-2 chain; peroxiredoxin 3; β-5 tubulin; p32 chain comprising administering to the organism an effective amount of one or more antagonistic miRNAs that bind to one or more endogenous miRNAs so as to reverse the inhibition of SPARC protein expression by the endogenous miRNA.

62. A therapeutic composition for the prophylaxis or therapy of an organism afflicted with cancer, restenosis, other proliferative disease, osteoporosis or wound healing, comprising an isolated nucleic acid for the expression in the cells of the organism an effective amount of one or more antagonistic miRNAs that reverse the inhibition of SPARC protein expression caused by the endogenous miRNA.

63. The therapeutic composition of claim 62, wherein the antagonistic miRNA is a synthetic nucleic acid or encoded by an isolated nucleic acid.

64. The therapeutic composition of claim 63, wherein the isolated nucleic acid is a vector selected from the group consisting of a plasmid, cosmid, phagemid, virus, and artificial chromosome.

65. The therapeutic composition of claim 64, wherein the isolated nucleic acid further comprises one or more in vivo expression control elements selected from the group consisting of a promoter, enhancer, RNA splicing signal, and combinations thereof.

66. The therapeutic composition of claim 64, wherein the antagonistic miRNA is from 10 nucleotides to 170 nucleotides in length and increases reporter activity expressed from a construct that encodes a transcript for the reporter fused to the SPARC mRNA 3′ untranslated region.

67. The therapeutic composition of claim 62, wherein the antagonistic miRNA is from 10 to 50 nucleotides in length.

68. The therapeutic composition of claim 66, wherein the antagonistic miRNA is at least 90% complementary to one or more of the sequences selected from the group consisting of: SEQ ID NOS: 1-41 and 44-83 and combinations thereof.

69. The therapeutic composition of claim 66, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the proliferative disease is selected from the group consisting of hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas.

70. An isolated nucleic acid comprising one or more in vivo expression control elements operatively linked to a reporter gene, wherein said reporter gene is upstream of all or a portion of a SPARC 3′ untranslated region, wherein upon transfection of the isolated nucleic acid into eukaryotic cells, the in vivo expression control elements result the production of an mRNA encoding the reporter upstream of the SPARC 3′ untranslated region.

71. The isolated nucleic acid of claim 70, wherein the isolated nucleic acid is a vector selected from the group consisting of a plasmid, cosmid, phagemid, virus, and artificial chromosome.

72. The isolated nucleic acid of claim 71, wherein the one or more in vivo expression control elements are selected from the group consisting of a promoter, enhancer, RNA splicing signal, and combinations thereof.

73. The isolated nucleic acid of claim 70, wherein the reporter gene encodes a luciferase protein.

74. A kit for the identification of SPARC expression modulators comprising:
(a) first isolated nucleic acid with a first set of one or more in vivo expression control elements operatively linked to a first reporter gene which is cloned upstream of all or a portion of a SPARC 3′ untranslated region, wherein upon transfection of said first isolated nucleic acid into eukaryotic cells, the first set of in vivo expression control elements result the production of an mRNA encoding the first reporter upstream of the SPARC 3′ untranslated region;
(b) a second isolated nucleic acid comprising said the set of in vivo expression control elements from (a) operatively linked to said first reporter gene, wherein upon transfection of said second isolated nucleic acid into eukaryotic cells, the in vivo expression control elements result in the transcription of an mRNA encoding said first reporter molecule; and
(c) a third isolated nucleic acid comprising a second set of one or more in vivo expression control elements operatively linked to a second reporter gene, wherein upon transfection of the isolated nucleic acid into eukaryotic cells, said second set of in vivo expression control elements result in the expression of said second reporter.

75. Method of identifying SPARC expression modulators comprising:
(a) transfecting eukaryotic cells with an isolated nucleic acid comprising one or more in vivo expression control elements operatively linked to a reporter gene which is cloned upstream of all or a portion of a SPARC 3′ untranslated region, wherein the in vivo expression control elements result the production of an mRNA encoding the reporter upstream of the SPARC 3′ untranslated region, and
(b) transfecting other eukaryotic cells with isolated nucleic acid comprising said one or more in vivo expression control elements operatively linked to said reporter gene, wherein the expression control elements result in the transcription of an mRNA encoding the reporter molecule,
(c) contacting and mock-contacting the transfected cells from (a) and (b) with a candidate expression modulator, and
(d) comparing the reporter gene activity in the transfected cells from (a) and (b) with and without contacting the transfected cells with candidate expression modulator.

76. The method of claim 75, further comprising the co-transfection of the cells in (a) and (b), with a second report construct expressing a second reporter for the normalization the data compared in (d).

77. The method of claim 75, further comprising mutating the SPARC 3′ untranslated region in the reporter expression construct, transfecting said mutated reporter expression construct into eukaryotic cells, and comparing the reporter gene activity resulting from expression of the mutated and unmutated reporter expression constructs with and without contacting the transfected cells with candidate expression modulator.

78. A SPARC expression modulator identified by the method of claim 75.

79. The SPARC expression modulator of claim 78, wherein said SPARC expression modulator is a small molecule, LNA, nucleic acid, peptide-nucleic acid, miRNA or a polypeptide.

80. The use of a miRNA as biomarker for proliferative disease progression, response to a treatment of proliferative disease or combinations thereof.

81. The method of 80, where miRNA is detected by RT-PCR, microarray, non-PCR nucleic acid detection assay or mass spectroscopy.


SEQUENCE LISTING (83 sequences)

SEQ ID NO: 1 - DNA, 3178 bp, Homo sapiens
gttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaaaaaaaaaaaaaaaaaaaaaaaaaaaaaaaaaaaaaaa

SEQ ID NO: 2 - RNA, 74 nt, Homo sapiens
ccgcacucucuccauuacacuacccugccucuucuccaugagaggcagcgggguguaguggauagagcacgggu

SEQ ID NO: 3 - RNA, 83 nt, Homo sapiens
cggggugagguaguagguugugugguuucagggcagugauguugccccucggaagauaacuauacaaccuacugccuucccug

SEQ ID NO: 4 - RNA, 84 nt, Homo sapiens
cuggcugagguaguaguuugugcuguuggucggguugugacauugcccgcuguggagauaacugcgcaagcuacugccuugcua

SEQ ID NO: 5 - RNA, 86 nt, Homo sapiens
ugcuuguaacuuuccaaagaauucuccuuuugggcuuucugguuuuauuuuaagcccaaaggugaauuuuuugggaaguuugagcu

SEQ ID NO: 6 - RNA, 89 nt, Homo sapiens
accagacuuuuccuagucccugagacccuaacuugugagguauuuuaguaacaucacaagucaggcucuugggaccuaggcggagggga

SEQ ID NO: 7 - RNA, 87 nt, Homo sapiens
ccuaggaagagguaguagguugcauaguuuuagggcagggauuuugcccacaaggagguaacuauacgaccugcugccuuucuuagg

SEQ ID NO: 8 - RNA, 77 nt, Homo sapiens
agucuaguuacuaggcaguguaguuagcugauugcuaauaguaccaaucacuaaccacacggccagguaaaaagauu

SEQ ID NO: 9 - RNA, 68 nt, Homo sapiens
guguauucuacagugcacgugucuccaguguggcucggaggcuggagacgcggcccuguuggaguaac

SEQ ID NO: 10 - RNA, 80 nt, Homo sapiens
ccuguugccacaaacccguagauccgaacuugugguauuaguccgcacaagcuuguaucuauagguaugugucuguuagg

SEQ ID NO: 11 - RNA, 84 nt, Homo sapiens
gugcucgguuuguaggcagugucauuagcugauuguacuguggugguuacaaucacuaacuccacugccaucaaaacaaggcac

SEQ ID NO: 12 - RNA, 84 nt, Homo sapiens
gcauccggguugagguaguagguuguaugguuuagaguuacacccugggaguuaacuguacaaccuucuagcuuuccuuggagc

SEQ ID NO: 13 - RNA, 64 nt, Homo sapiens
augacugauuucuuuugguguucagagucaauauaauuuucuagcaccaucugaaaucgguuau

SEQ ID NO: 14 - RNA, 64 nt, Homo sapiens; identical to SEQ ID NO: 13

SEQ ID NO: 15 - RNA, 81 nt, Homo sapiens
cuucaggaagcugguuucauauggugguuuagauuuaaauagugauugucuagcaccauuugaaaucaguguucuuggggg

SEQ ID NO: 16 - RNA, 81 nt, Homo sapiens
cuucuggaagcugguuucacaugguggcuuagauuuuuccaucuuuguaucuagcaccauuugaaaucaguguuuuaggag

SEQ ID NO: 17 - RNA, 88 nt, Homo sapiens
aucucuuacacaggcugaccgauuucuccugguguucagagucuguuuuugucuagcaccauuugaaaucgguuaugauguaggggga

SEQ ID NO: 18 - RNA, 84 nt, Homo sapiens
aggcugagguaguaguuuguacaguuugagggucuaugauaccacccgguacaggagauaacuguacaggccacugccuugcca

SEQ ID NO: 19 - RNA, 73 nt, Homo sapiens
ccuggcacugagaacugaauuccauaggcugugagcucuagcaaugcccuguggacucaguucuggugcccgg

SEQ ID NO: 20 - RNA, 84 nt, Homo sapiens
gugguacuugaagauagguuauccguguugccuucgcuuuauuugugacgaaucauacacgguugaccuauuuuucaguaccaa

SEQ ID NO: 21 - RNA, 100 nt, Homo sapiens
ggccuagucaucacccugagccuugcacugagaugggagugguguaaggcucagguaugcacagcucccaucucagaacaaggcucgggugugcucagcu

SEQ ID NO: 22 - RNA, 87 nt, Homo sapiens
ucagagugagguaguagauuguauaguugugggguagugauuuuacccuguucaggagauaacuauacaaucuauugccuucccuga

SEQ ID NO: 23 - RNA, 72 nt, Homo sapiens
ugucggguagcuuaucagacugauguugacuguugaaucucauggcaacaccagucgaugggcugucugaca

SEQ ID NO: 24 - RNA, 85 nt, Homo sapiens
ggcugagccgcaguaguucuucaguggcaagcuuuauguccugacccagcuaaagcugccaguugaagaacuguugcccucugcc

SEQ ID NO: 25 - RNA, 73 nt, Homo sapiens
ggccggcugggguuccuggggaugggauuugcuuccugucacaaaucacauugccagggauuuccaaccgacc

SEQ ID NO: 26 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 27 - RNA, 80 nt, Homo sapiens
ugggaugagguaguagguuguauaguuuuagggucacacccaccacugggagauaacuauacaaucuacugucuuuccua

SEQ ID NO: 28 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 29 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 30 - RNA, 89 nt, Homo sapiens
ugcugcuggccagagcucuuuucacauugugcuacugucugcaccugucacuagcagugcaauguuaaaagggcauuggccguguagug

SEQ ID NO: 31 - RNA, 110 nt, Homo sapiens
ucaccuggccaugugacuugugggcuucccuuugucauccuucgccuagggcucugagcagggcagggacagcaaaggggugcucaguugucacuucccacagcacggag

SEQ ID NO: 32 - RNA, 83 nt, Homo sapiens
ucucaugcagucauucuccaaaagaaagcacuuucuguugucugaaagcagagugccuucuuuuggagcguuacuguuugaga

SEQ ID NO: 33 - RNA, 87 nt, Homo sapiens
ucucaggcagugacccucuagauggaagcacugucuguuguauaaaagaaaagaucgugcaucccuuuagaguguuacuguuugaga

SEQ ID NO: 34 - RNA, 67 nt, Homo sapiens
gugacccucuagauggaagcacugucuguugucuaagaaaagaucgugcaucccuuuagaguguuac

SEQ ID NO: 35 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 36 - RNA, 21 nt, Homo sapiens
auguaugugugcaugugcaug

SEQ ID NO: 37 - RNA, 24 nt, Homo sapiens
cugaagugauguguaacugaucag

SEQ ID NO: 38 - RNA, 22 nt, Homo sapiens
uuugugaccugguccacuaacc

SEQ ID NO: 39 - RNA, 22 nt, Homo sapiens
uuugugaccugguccacuaacc

SEQ ID NO: 40 - RNA, 23 nt, Homo sapiens
uggaagacuagugauuuuguugu

SEQ ID NO: 41 - RNA, 22 nt, Homo sapiens
uggaauguaaagaaguauguau

SEQ ID NO: 42 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 43 - protein, 286 aa, Homo sapiens
APQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 44 - RNA, 22 nt, Homo sapiens
uccauuacacuacccugccucu

SEQ ID NO: 45 - RNA, 22 nt, Homo sapiens
ugagguaguagguugugugguu

SEQ ID NO: 46 - RNA, 22 nt, Homo sapiens
ugagguaguaguuugugcuguu

SEQ ID NO: 47 - RNA, 22 nt, Homo sapiens
caaagaauucuccuuuugggcu

SEQ ID NO: 48 - RNA, 22 nt, Homo sapiens
ucccugagacccuaacuuguga

SEQ ID NO: 49 - RNA, 22 nt, Homo sapiens
agagguaguagguugcauaguu

SEQ ID NO: 50 - RNA, 23 nt, Homo sapiens
aggcaguguaguuagcugauugc

SEQ ID NO: 51 - RNA, 22 nt, Homo sapiens
ucuacagugcacgugucuccag

SEQ ID NO: 52 - RNA, 22 nt, Homo sapiens
aacccguagauccgaacuugug

SEQ ID NO: 53 - RNA, 22 nt, Homo sapiens
caaucacuaacuccacugccau

SEQ ID NO: 54 - RNA, 22 nt, Homo sapiens
ugagguaguagguuguaugguu

SEQ ID NO: 55 - RNA, 22 nt, Homo sapiens
uagcaccaucugaaaucgguua

SEQ ID NO: 56 - RNA, 22 nt, Homo sapiens
acugauuucuuuugguguucag

SEQ ID NO: 57 - RNA, 23 nt, Homo sapiens
uagcaccauuugaaaucaguguu

SEQ ID NO: 58 - RNA, 23 nt, Homo sapiens
uagcaccauuugaaaucaguguu

SEQ ID NO: 59 - RNA, 22 nt, Homo sapiens
uagcaccauuugaaaucgguua

SEQ ID NO: 60 - RNA, 22 nt, Homo sapiens
ugagguaguaguuuguacaguu

SEQ ID NO: 61 - RNA, 22 nt, Homo sapiens
ugagaacugaauuccauaggcu

SEQ ID NO: 62 - RNA, 22 nt, Homo sapiens
uagguuauccguguugccuucg

SEQ ID NO: 63 - RNA, 22 nt, Homo sapiens
gcacugagaugggaguggugua

SEQ ID NO: 64 - RNA, 22 nt, Homo sapiens
ugagguaguagauuguauaguu

SEQ ID NO: 65 - RNA, 22 nt, Homo sapiens
uagcuuaucagacugauguuga

SEQ ID NO: 66 - RNA, 22 nt, Homo sapiens
aagcugccaguugaagaacugu

SEQ ID NO: 67 - RNA, 21 nt, Homo sapiens
aucacauugccagggauuucc

SEQ ID NO: 68 - RNA, 22 nt, Homo sapiens
ugagguaguaaguuguauuguu

SEQ ID NO: 69 - RNA, 22 nt, Homo sapiens
ugagguaguagguuguauaguu

SEQ ID NO: 70 - RNA, 22 nt, Homo sapiens
acaguagucugcacauugguua

SEQ ID NO: 71 - RNA, 22 nt, Homo sapiens
acagcaggcacagacaggcagu

SEQ ID NO: 72 - RNA, 22 nt, Homo sapiens
cagugcaauguuaaaagggcau

SEQ ID NO: 73 - RNA, 22 nt, Homo sapiens
uucccuuugucauccuucgccu

SEQ ID NO: 74 - RNA, 24 nt, Homo sapiens
uucuccaaaagaaagcacuuucug

SEQ ID NO: 75 - RNA, 22 nt, Homo sapiens
aucgugcaucccuuuagagugu

SEQ ID NO: 76 - RNA, 22 nt, Homo sapiens
ucgugcaucccuuuagaguguu

SEQ ID NO: 77 - RNA, 22 nt, Homo sapiens
gugaaauguuuaggaccacuag

SEQ ID NO: 78 - RNA, 21 nt, Homo sapiens
auguaugugugcaugugcaug

SEQ ID NO: 79 - RNA, 24 nt, Homo sapiens
cugaagugauguguaacugaucag

SEQ ID NO: 80 - RNA, 22 nt, Homo sapiens
uuugugaccugguccacuaacc

SEQ ID NO: 81 - RNA, 21 nt, Homo sapiens
caaagaggaaggucccauuac

SEQ ID NO: 82 - RNA, 23 nt, Homo sapiens
uggaagacuagugauuuuguugu

SEQ ID NO: 83 - RNA, 22 nt, Homo sapiens
uggaauguaaagaaguauguau